(19) United States
(12) Patent Application Publication
(10) Pub. No.: US 2023/0140014 A1
(43) Pub. Date: May 4, 2023

(54) Methods and Systems for Prophylactically Preventing, Slowing the Progression of, or Treating Cerebral Amyloid Angiopathy, Alzheimer’s Disease, and/or Acute Stroke

(71) Applicant: HDL Therapeutics, Inc., Vero Beach, FL (US)

(72) Inventors: Hollis Bryan Brewer, JR., Potomac, MD (US); Michael M. Matin, Short Hills, NJ (US)

(21) Appl. No.: 18/045,920

(22) Filed: Oct. 12, 2022

Related U.S. Application Data
(63) Continuation-in-part of application No. 17/571,225, filed on Jan. 7, 2022.
Application No. 17/571,225 is a continuation-in-part of application No. 17/315,509, filed on May 10, 2021.
Application No. 17/315,509 is a continuation of application No. 17/021,883, filed on Sep. 15, 2020, now Pat. No. 11,033,582.
Application No. 17/021,883 is a continuation of application No. 16/225,210, filed on Dec. 19, 2018, now Pat. No. 10,821,133.
Application No. 17/571,225 is a continuation-in-part of application No. 17/012,410, filed on Sep. 4, 2020.
Application No. 17/012,410 is a continuation of application No. 16/198,672, filed on Nov. 21, 2018, now Pat. No. 11,027,052.
Application No. 17/571,225 is a continuation-in-part of application No. 17/012,396, filed on Sep. 4, 2020, now Pat. No. 11,400,188.
Application No. 17/012,396 is a continuation of application No. 16/198,672, filed on Nov. 21, 2018, now Pat. No. 11,027,052.
Application No. 17/571,225 is a continuation-in-part of application No. 16/698,193, filed on Nov. 27, 2019, now abandoned.
Application No. 16/698,193 is a continuation-in-part of application No. 16/409,543, filed on May 10, 2019, now abandoned.
Application No. 16/409,543 is a continuation-in-part of application No. 15/909,765, filed on Mar. 1, 2018, now abandoned.
Application No. 15/909,765 is a continuation-in-part of application No. 15/876,808, filed on Jan. 22, 2018.
Application No. 16/698,193 is a continuation-in-part of application No. 16/225,210, filed on Dec. 19, 2018, now Pat. No. 10,821,133.
Application No. 16/698,193 is a continuation-in-part of application No. 16/198,672, filed on Nov. 21, 2018, now Pat. No. 11,027,052.
Application No. 16/698,193 is a continuation-in-part of application No. 16/046,830, filed on Jul. 26, 2018, now abandoned.
Application No. 16/046,830 is a continuation-in-part of application No. 15/909,765, filed on Mar. 1, 2018, now abandoned.
Application No. 17/571,225 is a continuation-in-part of application No. 16/409,543, filed on May 10, 2019, now abandoned.
Application No. 17/571,225 is a continuation-in-part of application No. 16/046,830, filed on Jul. 26, 2018, now abandoned.
Application No. 17/571,225 is a continuation-in-part of application No. 15/909,765, filed on Mar. 1, 2018, now abandoned.
Application No. 17/571,225 is a continuation-in-part of application No. 15/876,808, filed on Jan. 22, 2018.
(60) Provisional application No. 63/296,928, filed on Jan. 6, 2022.
(60) Provisional application No. 63/262,423, filed on Oct. 12, 2021.
(60) Provisional application No. 63/135,001, filed on Jan. 8, 2021.
(60) Provisional application No. 62/611,098, filed on Dec. 28, 2017.
(60) Provisional application No. 62/589,919, filed on Nov. 22, 2017.
(60) Provisional application No. 62/773,388, filed on Nov. 30, 2018.
(60) Provisional application No. 62/700,804, filed on Jul. 19, 2018.
(60) Provisional application No. 62/670,615, filed on May 11, 2018.
(60) Provisional application No. 62/537,581, filed on Jul. 27, 2017.
(60) Provisional application No. 62/537,881, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.

(51) Int. Cl. A61K 35/16; A61K 38/17

(52) U.S. Cl. CPC A61K 35/16; A61K 38/17

(57) ABSTRACT

Systems, apparatuses and methods for prophylactically preventing, or for treating the onset and/or progression of Cerebral Amyloid Angiopathy (CAA), acute stroke conditions, or Alzheimer’s disease include the administration to a patient pre-beta HDL particles, a CETP inhibitor, or a combination of both pre-beta HDL particles and a CETP inhibitor. The progression of, stabilizing, or improving symptoms related to these conditions are treated by monitoring a pathophysiological change indicative of the conditions in a patient, based on the monitoring, determining if amyloid plaque is present in a perivascular space/IPAD System/Perivascular Pathway of the patient, optionally determining an extent of amyloid plaque in the perivascular space/IPAD System/Perivascular Pathway, and based on the presence of amyloid plaque in the perivascular space/IPAD System/Perivascular Pathway of the patient, determining a treatment protocol for the patient. The treatment protocol includes administering to the patient a high density lipoprotein composition derived from mixing a blood fraction with a lipid removing agent, wherein the high density lipoprotein composition comprises pre-beta HDL, administering to the patient a CETP inhibitor, or administering to the patient both the high density lipoprotein composition and the CETP inhibitor.

### CROSS-REFERENCE

The present application relies on U.S. Pat. Provisional Application No. 63/262,423, entitled “Methods for Prophylactically Preventing, Slowing the Progression of, or Treating Cerebral Amyloid Angiopathy, Alzheimer’s Disease and/or Acute Stroke” and filed on Oct. 12, 2021, for priority, which is herein incorporated by reference in its entirety.

The present application also relies on U.S. Pat. Provisional Application No. 63/296,928, titled “System and Method for Reducing Low Attenuation Plaque and/or Plaque Burden in Patients”, filed on Jan. 6, 2022, for priority, which is herein incorporated by reference in its entirety.

The present application is also a continuation-in-part application of U.S. Pat. Application No. 17/571,225, entitled “Systems and Methods for Reducing Low Attenuation Plaque and/or Plaque Burden in Patients” and filed on Jan. 7, 2022, which relies on, for priority, U.S. Pat. Provisional Application No. 63/296,928, titled “System and Method for Reducing Low Attenuation Plaque and/or Plaque Burden in Patients”, filed on Jan. 6, 2022, U.S. Pat. Provisional Application No. 63/262,423, titled “Methods for Prophylactically Preventing, Slowing the Progression of, or Treating Cerebral Amyloid Angiopathy, Alzheimer’s Disease and/or Acute Stroke”, filed on Oct. 12, 2021, and U.S. Pat. Provisional Application No. 63/135,001, titled “Methods for Treating Lipid-Related Dysfunction in Lupus Patients”, filed on Jan. 8, 2021.

U.S. Pat. Application No. 17/571,225 is also a continuation-in-part application of United States Patent Application No. 17/315,509, titled “Methods for Preserving and Administering Pre-Beta High Density Lipoprotein Having a Predetermined Minimum Level of Degradation” and filed on May 10, 2021, which is a continuation application of U.S. Pat. Application No. 17/021,883, of the same title, filed on Sep. 15, 2020, and issued as U.S. Pat. No. 11,033,582 on Jun. 15, 2021, which is a continuation application of U.S. Pat. Application No. 16/225,210, titled “Methods for Preserving and Administering Pre-Beta High Density Lipoprotein Extracted from Human Plasma”, filed on Dec. 19, 2018, and issued as U.S. Pat. No. 10,821,133 on Nov. 3, 2020, which, in turn, relies on, for priority, U.S. Provisional Pat. Application Number 62/611,098, titled “Methods for Treating Cholesterol-Related Diseases” and filed on Dec. 28, 2017.

U.S. Pat. Application No. 17/571,225 is also a continuation-in-part application of U.S. Pat. Application No. 17/012,410, titled “Systems for Removing Air from the Fluid Circuits of a Plasma Processing System” and filed on Sep. 4, 2020, which is a continuation application of U.S. Pat. Application No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which, in turn, relies on U.S. Provisional Pat. Application No. 62/589,919, entitled “Systems and Methods for Causing Regression of Arterial Plaque” and filed on Nov. 22, 2017, for priority.

U.S. Pat. Application No. 17/571,225 is also a continuation-in-part application of U.S. Pat. Application No. 17/012,396, titled “Systems for Removing Air from the Fluid Circuits of a Plasma Processing System”, filed on Sep. 4, 2020, and issued as U.S. Pat. No. 11,400,188 on Aug. 2, 2022, which is a continuation application of U.S. Pat. Application No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which, in turn, relies on U.S. Provisional Pat. Application No. 62/589,919, entitled “Systems and Methods for Causing Regression of Arterial Plaque” and filed on Nov. 22, 2017, for priority.

U.S. Pat. Application No.17/571,225 is also a continuation-in-part application of U.S. Pat. Application No. 16/698,193, titled “Methods for Treating Lipid-Related Diseases Including Xanthomas, Carotid Artery Stenoses, and Cerebral Atherosclerosis” and filed on Nov. 27, 2019, which relies on U.S. Pat. Provisional Application No. 62/773,388, titled “Methods for Treating Cholesterol Related Diseases” and filed on Nov. 30, 2018, for priority.

U.S. Pat. Application No.16/698,193 is also a continuation-in-part application of U.S. Pat. Application No. 16/409,543, titled “Methods for Prophylactically Preventing, Slowing the Progression of, or Treating Cerebral Amyloid Angiopathy, Alzheimer’s Disease and/or Acute Stroke” and filed on May 10, 2019, which relies on U.S. Provisional Pat. Application No. 62/700,804, titled “Methods for Prophylactically Preventing, Slowing the Progression of, or Treating Cerebral Amyloid Angiopathy, Alzheimer’s Disease and/or Acute Stroke” and filed on Jul. 19, 2018 and U.S. Pat. Application No. 62/670,615, of the same title and filed on May 11, 2018, for priority.

U.S. Pat. Application No. 16/409,543 is also a continuation-in-part application of U.S. Pat. Application No. 15/909,765, titled “Methods for Prophylactically Preventing, Slowing the Progression of, or Treating Alzheimer’s Disease” and filed on Mar. 1, 2018, which relies on U.S. Pat. Application No. 62/537,581, titled “Methods for Treating Cholesterol-Related Diseases” and filed on Jul. 27, 2017, U.S. Pat. Application No. 62/516,100, entitled “Methods for Treating Cholesterol-Related Diseases” and filed on Jun. 6, 2017, and U.S. Provisional Pat. Application No. 62/465,262, entitled “Method for Treating Familial Hypercholesterolemia” and filed on Mar. 1, 2017, for priority.

U.S. Pat. Application No.r 15/909,765 is also a continuation-in-part application of U.S. Pat. Application No. 15/876,808, titled “Methods for Treating Cholesterol-Related Diseases” and filed on Jan. 22, 2018, which, in turn, relies on U.S. Provisional Pat. Application No. 62/516,100, titled “Methods for Treating Cholesterol-Related Diseases” and filed on Jun. 6, 2017, U.S. Provisional Pat. Application No. 62/465,262, titled “Method for Treating Familial Hypercholesterolemia” and filed on Mar. 1, 2017, and U.S. Provisional Pat. Application No. 62/449,416, titled “Method for Treating Familial Hypercholesterolemia” and filed on Jan. 23, 2017, for priority.

U.S. Pat. Application No. 16/698,193 is also a continuation-in-part application of United States Patent Application Number 16/225,210, entitled “Methods for Preserving and Administering Pre-Beta High Density Lipoprotein Extracted from Human Plasma”, filed on Dec. 19, 2018, and issued as U.S. Pat. No. 10,821,133 on Nov. 3, 2020, which relies on U.S. Provisional Pat. Application No.r 62/611,098, titled “Methods for Treating Cholesterol-Related Diseases” and filed on Dec. 28, 2017, for priority.

U.S. Pat. Application No. 16/698,193 is also a continuation-in-part application of U.S. Pat. Application No. 16/198,672, titled “Systems and Methods for Priming Fluid Circuits of a Plasma Processing System”, filed on Nov. 21, 2018, and issued as U.S. Pat. No. 11,027,052 on Jun. 8, 2021, which relies on U.S. Provisional Pat. Application No. 62/589,919, titled “Systems and Methods for Causing Regression of Arterial Plaque” and filed on Nov. 22, 2017, for priority.

U.S. Pat. Application No. 16/698,193 is also a continuation-in-part application of U.S. Pat. Application No. 16/046,830, titled “Methods for Treating Cholesterol-Related Diseases Using Administered Solutions Having Increased Pre-Beta HDL Particles” and filed on Jul. 26, 2018, which relies on U.S. Provisional Pat. Application No. 62/537,581, titled “Method for Treating Cholesterol-Related Diseases” and filed on Jul. 27, 2017, for priority.

U.S. Pat. Application No. 16/046,830 is also a continuation-in-part application of U.S. Pat. Application No. 15/909,765, entitled “Methods for Prophylactically Preventing, Slowing the Progression of, or Treating Alzheimer’s Disease”, and filed on Mar. 1, 2018, which, in turn, relies on U.S. Provisional Pat. Application No. 62/537,581, entitled “Method for Treating Cholesterol-Related Diseases” and filed on Jul. 27, 2017, U.S. Provisional Pat. Application No. 62/516,100, entitled “Methods for Treating Cholesterol-Related Diseases” and filed on Jun. 6, 2017, and U.S. Provisional Pat. Application No. 62/465,262, entitled “Method for Treating Familial Hypercholesterolemia” and filed on Mar. 1, 2017, for priority.

U.S. Pat. Application No. 17/571,225 is also a continuation-in-part application of U.S. Pat. Application No. 16/409,543, titled “Methods for Prophylactically Preventing, Slowing the Progression of, or Treating Cerebral Amyloid Angiopathy, Alzheimer’s Disease and/or Acute Stroke” and filed on May 10, 2019, which relies on U.S. Provisional Pat. Application No. 62/700,804, titled “Methods for Prophylactically Preventing, Slowing the Progression of, or Treating Cerebral Amyloid Angiopathy, Alzheimer’s Disease and/or Acute Stroke” and filed on Jul. 19, 2018 and U.S. Provisional Pat. Application No. 62/670,615, of the same title and filed on May 11, 2018, for priority.

U.S. Pat. Application No. 17/571,225 is also a continuation-in-part application of U.S. Pat. Application No. . 16/046,830, tilted “Methods for Treating Cholesterol-Related Diseases Using Administered Solutions Having Increased Pre-Beta HDL Particles” and filed on Jul. 26, 2018, which relies on U.S. Provisional Pat. Application No. 62/537,581, entitled “Method for Treating Cholesterol-Related Diseases” and filed on Jul. 27, 2017, for priority.

U.S. Pat. Application No. 17/571,225 is also a continuation-in-part application of U.S. Pat. Application No. 15/909,765, entitled “Methods for Prophylactically Preventing, Slowing the Progression of, or Treating Alzheimer’s Disease” and filed on Mar. 1, 2018, which relies on U.S. Provisional Patent Application No. 62/465,262, entitled “Method for Treating Familial Hypercholesterolemia” and filed on Mar. 1, 2017, U.S. Provisional Pat. Application No. 62/516,100, entitled “Methods for Treating Cholesterol-Related Diseases” and filed on June 6, and U.S. Provisional Pat. Application No. 62/537,581, entitled “Methods for Treating Cholesterol-Related Diseases” and filed on Jul. 27, 2017, for priority.

U.S. Pat. Application No. 17/571,225 is also a continuation-in-part application of U.S. Pat. Application No. 15/876,808, titled “Methods for Treating Cholesterol-Related Diseases”, and filed on Jan. 22, 2018, which, in turn, relies on U.S. Provisional Pat. Application No. 62/516,100, entitled “Methods for Treating Cholesterol-Related Diseases” and filed on Jun. 6, 2017, U.S. Provisional Pat. Application No. 62/465,262, entitled “Method for Treating Familial Hypercholesterolemia” and filed on Mar. 1, 2017, and U.S. Provisional Pat. Application No. 62/449,416, entitled “Method for Treating Familial Hypercholesterolemia” and filed on Jan. 23, 2017, for priority.

The above-mentioned applications are herein incorporated by reference in their entirety.

### FIELD

The method of the present specification provides for either an isolated or a successively repeated treatment procedure for selective removal of lipid from HDL to create a pre-beta HDL particle while leaving LDL particles substantially intact and the administration of the pre-beta HDL particle to an individual having an acute stroke, or cerebral cognitive impairment with Alzheimer’s disease, in order to treat, delay, halt and stabilize, reverse or improve the progression of the disease or pathophysiologic process that leads to the symptoms related to acute stroke or Alzheimer’s disease.

### BACKGROUND

Cerebral Amyloid Angiopathy (CAA) is an aging-related condition caused by deposits of amyloid proteins in the wall or perivascular space (such as in the intramural peri-arterial drainage (IPAD) System/Perivascular Pathway) of blood vessels in a brain. Low levels of CAA may usually be harmless, however, severe CAA may lead to the protein deposits causing the blood vessels to crack, in which case the blood can leak out and damage the brain. Amyloids are similar to the deposits in the brain that cause Alzheimer’s disease (AD). The factors known to increase risks of CAA include advancing age, an accompanying presence of AD, and some type of genes. Specifically, the gene known as Apolipoprotein E is considered to be a risk factor for CAA. CAA is also estimated to be the cause of 30-40% of hemorrhagic strokes. Differential diagnosis may be performed to determine the probability of CAA in a patient. Imaging tests like CT scans or MRI scans can show whether a bleeding occurred in the outer part of the brain (the cortex) where CAA is usually most severe. This can help distinguish CAA from hemorrhagic strokes caused by high blood pressure, which tend to occur in deep sections of the brain. In addition, a kind of MRI scan called gradient-echo MRI can show whether there have been other tiny areas of bleeding that are also in the typical locations for CAA.

The deposits of amyloid proteins in the arteries and blood vessels of the brain, in association with an appropriate genetic cause, may also result in Hereditary CAA (HCAA) or Hereditary Cerebral Hemorrhage with Amyloidosis (HCHWA). There are many different types of HCAA and HCHWA that are currently known. The different types are distinguished by their genetic cause and the signs and symptoms that occur. Mutations in the relevant gene lead to the production of proteins that are less stable than normal and that tend to cluster together (aggregate). These aggregated proteins form protein clumps called amyloid deposits that accumulate in certain areas of the brain and in its blood vessels. The amyloid deposits, known as plaques, damage brain cells, eventually causing cell death and impairing various parts of the brain. Brain cell loss in people with HCAA or HCHWA can lead to seizures, movement abnormalities, and other neurological problems. In blood vessels, amyloid plaques replace the muscle fibers and elastic fibers that give the blood vessels flexibility, causing them to become weak and prone to breakage. A break in a blood vessel in the brain causes bleeding in the brain (hemorrhagic stroke), which can lead to brain damage and dementia.

The protein deposits in CAA are usually characterized by amyloid beta peptide deposits within small to medium-sized blood vessels of the brain and leptomeninges. It is believed that vascular amyloid deposits in sporadic CAA are biochemically similar to the material comprising senile plaques in Alzheimer’s disease (AD). Sometimes, CAA occurs either in association with AD or as a certain familial syndrome such as familial hypercholesterolemia. The ratio of certain amyloid peptides in plasma or cerebrospinal fluid, particularly beta amyloid peptides 42 and 40, may be used as an indicator of the presence of CAA and/or AD in a patient. For example, a healthy individual may have levels, in the plasma, of beta amyloid peptide 42 of approximately 19.6 pg/ml and of beta amyloid peptide 40 of approximately 276.7 pg/ml, for a ratio of beta amyloid peptide 42 to beta amyloid peptide 40 of approximately 0.073. A patient with CAA or AD may have levels, in the plasma, of beta amyloid peptide 42 of approximately 13.2 pg/ml and of beta amyloid peptide 40 of approximately 244.3 pg/ml, for a ratio of beta amyloid peptide 42 to beta amyloid peptide 40 of approximately 0.057. Therefore, CAA and AD patients present with a decreased ratio of beta amyloid peptide 42 to beta amyloid peptide 40 in the plasma compared to individuals without CAA or AD.

Recent studies have indicated that, in addition to amyloid beta peptides produced in the brain, a significant amount of amyloid beta peptides may be produced in the periphery. These peripherally produced amyloid beta peptides may be produced in the liver or outside of the liver and are present in the plasma peripheral to the brain. The peripherally produced amyloid beta peptides are sufficiently small to travel through the patient’s circulatory system, cross the blood brain barrier, and then become stuck or deposited in the brain, forming amyloid plaques and causing cerebrovascular disease as discussed above.

Historically, the use of clinical criteria that defined later stages of Alzheimer’s disease (AD), such as after the onset of severe and marked dementia, determined the patients that were enrolled in clinical trials exhibited both the cognitive changes typical of clinically evident AD and the degree of functional impairment associated with marked dementia. As the scientific understanding of AD has evolved, efforts have been made to incorporate additional diagnostic information in order to enroll a greater class of patients in clinical trials. This diagnostic information includes, to varying degrees, the use of biomarkers reflecting underlying pathophysiological changes, which allows for the enrollment of patients in which there may be no apparent functional impairment or no detectable clinical abnormality. These patients are categorized as early onset AD patients. In using a broader range of diagnostic information to assess the degree and extent of AD, it is possible to intervene much earlier in the disease process given the onset of pathophysiological changes that can be measured and that precede clinically evident findings. There is thus a need to delay, halt, and preferably reverse the pathophysiological process that leads to the initial clinical deficits presented by AD.

AD is determined using results from several tests to arrive at a differential diagnosis. Thus, there is no definitive diagnosis for AD. Research has indicated that familial hypercholesterolemia is an early risk factor for AD. It is theorized that LDL receptors are involved in increasing the risk of AD. It has been observed that certain individuals are predisposed to AD, as demonstrated by family history or by genetic testing. Given that there is no established treatment for AD once lesions are formed, it would be desirable to provide a prophylactic way to treat AD or prevent the onset of AD altogether.

Accumulating epidemiological, pathological and imaging evidence over several decades has suggested a role for cerebrovascular disease in the onset and progression of AD. Brains of many people with AD have vascular microinfarcts (not usually detected by MRI), white matter lesions, or vessel wall alterations, with Cerebral Amyloid Angiopathy (CAA) present in over 90% of cases with AD. Vascular risk factors, such as high blood pressure or cholesterol, obesity, elevated homocysteine, atherosclerosis, carotid stenosis, atrial fibrillation, diabetes, and coronary disease, are risk factors for AD in many epidemiological studies. Such findings have led to a bi-modal neurovascular model of AD, in which chronic hypoperfusion, failure of drainage of fluid and proteins and uncoupling of blood flow to brain metabolic demands over decades trigger neurodegeneration and amyloidogenesis. Furthermore, CAA may be implicated as a complication of amyloid-beta (Aβ) immunotherapies, most likely due to a failure of fluid drainage and/or plaque-derived, solubilized Aβ after immunotherapy, which suggests that facilitating the clearance of Aβ is an urgent necessary step in therapeutic strategies.

Existing apheresis and extracorporeal systems for treatment of plasma constituents and therefore lipid-related diseases suffer from a number of disadvantages that limit their ability to be used in clinical applications. A need exists for improved systems, apparatuses and methods capable of removing lipids from blood components in order to provide treatments and preventative measures for AD.

In addition to Alzheimer’s disease, vascular dementia creates changes in cognitive functioning (which is why it is sometimes referred to as “vascular cognitive impairment”) that is usually a result of a stroke that blocks major brain blood vessels.

One form of acute stroke, as mentioned above, is hemorrhagic stroke. Among 30-40% of hemorrhagic strokes in the elderly are known to be caused by CAA. CAA occurs due to deposits of protein in the wall of blood vessels in the brain. In cases of severe CAA, protein deposits cause the blood vessel wall to crack, resulting in leakage of blood, which damages the brain and causes hemorrhagic stroke.

Ischemic stroke occurs when there is a blockage in an artery leading to the brain, and may be a secondary condition caused by a hemorrhagic stroke. Among the different types of ischemic stroke, the most common are thrombotic and embolic. A thrombotic stroke occurs when diseased or damaged cerebral arteries become blocked by the formation of a blood clot within the brain. Cerebral thrombosis (thrombotic stroke) can also be divided into an additional two categories that correlate to the location of the blockage within the brain: large-vessel thrombosis and small-vessel thrombosis. Large-vessel thrombosis is the term used when the blockage is in one of the brain’s larger blood-supplying arteries such as the carotid or middle cerebral, while small-vessel thrombosis involves one (or more) of the brain’s smaller, yet deeper, penetrating arteries. This latter type of stroke is also called a lacuner stroke. An embolic stroke is also caused by a clot within an artery, but in this case the clot (or emboli) forms somewhere other than in the brain itself.

While the methods to selectively delipidate HDL particles overcomes several of the limitations stated above, what is also needed is a method to selectively remove lipid from HDL particles and thereby create pre-beta HDL particles with increased capacity to accept cholesterol, without substantially affecting LDL particles, in acute and chronic diseases. What is also needed is a method to successively monitor effectiveness of the pre-beta HDL particles in accepting cholesterol as well as amyloid particles in order to monitor the progress of a treatment using imaging techniques such as CT Angiography. Additionally, what is needed is a method to treat CAA, acute stroke and AD, or prevent the increase of CAA, and onset of acute stroke or AD.

What are also needed are systems and methods to effectively capture amyloid beta peptides before they cross the blood brain barrier and become trapped in the brain. These systems and methods would bind the amyloid beta peptides while still in the peripheral vascular system and transport them to a site of degradation, for example, the liver, to help in the prevention of cerebrovascular disease.

### SUMMARY

The following embodiments and aspects thereof are described and illustrated in conjunction with systems, tools and methods, which are meant to be exemplary and illustrative, not limiting in scope.

The present specification discloses a method for delaying a progression of, stabilizing, or improving symptoms related to cerebral amyloid angiopathy (CAA) in a patient, comprising: monitoring a pathophysiological change indicative of CAA in a patient; based on said monitoring, determining if at least one of amyloid plaque, Tau oligomers, or other oligomers is present in a perivascular space/IPAD System/Perivascular Pathway of the patient in excess of a predetermined threshold; based on a presence of the at least one of the amyloid plaque, the Tau oligomers, or the other oligomers in the perivascular space/IPAD System/Perivascular Pathway of the patient, determining a treatment protocol for the patient, wherein the treatment protocol comprises, at least in part, administering to the patient a high density lipoprotein composition derived from mixing a blood fraction with a lipid removing agent and administering to the patient a CETP inhibitor.

Optionally, the high density lipoprotein composition is adapted to facilitate a drainage of at least one of soluble beta amyloids, soluble Tau oligomers, or other soluble oligomers. Optionally, the method further comprises determining an amount of at least one of the amyloid plaque, the Tau oligomers, or the other oligomers in the perivascular space/IPAD System/Perivascular Pathway. Optionally, the method further comprises using diagnostic imaging to determine at least one of the presence or the extent of the at least one of the amyloid plaque, the Tau oligomers, or the other oligomers in the perivascular space/IPAD System/Perivascular Pathway of the patient.

Optionally, the drainage is facilitated via the patient’s intramural peri-arterial drainage (IPAD) pathway.

Optionally, the high density lipoprotein composition is derived by: obtaining the blood fraction from the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient. Optionally, the method further comprises: connecting the patient to a device for withdrawing blood; withdrawing blood from the patient; and separating blood cells from the blood to yield the blood fraction containing high density lipoproteins and low density lipoproteins.

Optionally, the pre-beta high density lipoproteins have an increased concentration of pre-beta high density lipoproteins relative to the high density lipoproteins from the blood fraction prior to mixing.

Optionally, the pre-beta high density lipoproteins have a concentration of alpha high density lipoproteins in addition to pre-beta high density lipoproteins from the blood fraction prior to mixing.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour +/- 100 mL/hr.

Optionally, the pathophysiological change is indicated by an accumulation of plaque in the perivascular space/IPAD System/Perivascular Pathway of the patient resulting in cerebral amyloid angiopathy.

Optionally, the method further comprises determining a severity of CAA in the patient using at least one of global functioning, cognitive functioning, activities of daily living, or behavioral assessments.

Optionally, after administering to the patient the high density lipoprotein composition and the CETP inhibitor, the patient experiences a decrease in an accumulation of the at least one of the amyloid plaque, the Tau oligomers, or other oligomers in the perivascular space/IPAD System/Perivascular Pathway.

Optionally, after administering to the patient the high density lipoprotein composition and the CETP inhibitor, a rate of degeneration of the patient’s physiological and/or cognitive parameters indicative of CAA decreases.

Optionally, after administering to the patient the high density lipoprotein composition and the CETP inhibitor, a rate of degeneration of the patient’s physiological and/or cognitive parameters indicative of CAA, slows down relative to a rate of degeneration of the patient’s physiological and/or cognitive parameters indicative of CAA before administering to the patient the high density lipoprotein composition and the CETP inhibitor.

Optionally, after administering to the patient the high density lipoprotein composition and the CETP inhibitor, the patient’s physiological and/or cognitive symptoms indicative of CAA improve relative to the patient’s physiological and/or cognitive symptoms indicative of CAA before administering to the patient the high density lipoprotein composition and the CETP inhibitor.

Optionally, the high density lipoprotein composition is derived by: obtaining a blood fraction from an individual other than the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the lipid removing agent is at least one of phenols, hydrocarbons, amines, ethers, esters, alcohols, halohydrocarbons, halocarbons, di-isopropyl ether (DIPE), diethyl ether (DEE), n-butanol, ethyl acetate, dichloromethane, chloroform, isoflurane, sevoflurane, perfluorocyclohexanes, trifluoroethane, cyclofluorohexanol, or combinations thereof.

Optionally, the CETP inhibitor is administered to the patient in a dose ranging from 1 mg to 1000 mg or any increment therein.

Optionally, the CETP inhibitor is administered to the patient at any time interval that will achieve the desired therapeutic outcome.

Optionally, the CETP inhibitor is administered to the patient in a dose ranging selected from one of 30 mg/day, 100 mg/day, or 500 mg/day.

The present specification also discloses a method for delaying a progression of, stabilizing, or improving symptoms related to cerebral amyloid angiopathy (CAA) in a patient, comprising: monitoring a pathophysiological change indicative of CAA in a patient; based on said monitoring, determining if at least one of amyloid plaque, Tau oligomers, or other oligomers is present in a perivascular space/IPAD System/Perivascular Pathway of the patient in excess of a predetermined threshold; based on a presence of the at least one of the amyloid plaque, the Tau oligomers, or the other oligomers in the perivascular space/IPAD System/Perivascular Pathway of the patient, determining a treatment protocol for the patient, wherein the treatment protocol comprises, at least in part, administering to the patient a CETP inhibitor.

Optionally, the method further comprises determining an amount of at least one of the amyloid plaque, the Tau oligomers, or the other oligomers in the perivascular space/IPAD System/Perivascular Pathway. Optionally, the method further comprises using diagnostic imaging to determine at least one of the presence or the extent of the at least one of the amyloid plaque, the Tau oligomers, or the other oligomers in the perivascular space/IPAD System/Perivascular Pathway of the patient.

Optionally, the drainage is facilitated via the patient’s intramural peri-arterial drainage (IPAD) pathway.

Optionally, the pathophysiological change is indicated by an accumulation of plaque in the perivascular space/IPAD System/Perivascular Pathway of the patient resulting in cerebral amyloid angiopathy.

Optionally, the method further comprises determining a severity of CAA in the patient using at least one of global functioning, cognitive functioning, activities of daily living, or behavioral assessments.

Optionally, after administering to the patient the CETP inhibitor, the patient experiences a decrease in an accumulation of the at least one of the amyloid plaque, the Tau oligomers, or other oligomers in the perivascular space/IPAD System/Perivascular Pathway.

Optionally, after administering to the patient the CETP inhibitor, a rate of degeneration of the patient’s physiological and/or cognitive parameters indicative of CAA decreases.

Optionally, after administering to the patient the CETP inhibitor, a rate of degeneration of the patient’s physiological and/or cognitive parameters indicative of CAA, slows down relative to a rate of degeneration of the patient’s physiological and/or cognitive parameters indicative of CAA before administering to the patient the CETP inhibitor.

Optionally, after administering to the patient the CETP inhibitor, the patient’s physiological and/or cognitive symptoms indicative of CAA improve relative to the patient’s physiological and/or cognitive symptoms indicative of CAA before administering to the patient the CETP inhibitor.

Optionally, the CETP inhibitor is administered to the patient in a dose ranging from 1 mg to 1000 mg.

Optionally, the CETP inhibitor is administered to the patient at any time interval that will achieve the desired therapeutic outcome.

Optionally, the CETP inhibitor is administered to the patient in a dose ranging selected from one of 30 mg/day, 100 mg/day, or 500 mg/day.

The present specification also discloses a method for delaying a progression of, stabilizing, or improving symptoms related to cerebral amyloid angiopathy (CAA) in a patient, comprising: monitoring a pathophysiological change indicative of CAA in a patient; based on said monitoring, determining if at least one of amyloid plaque, Tau oligomers, or other oligomers is present in a perivascular space/IPAD System/Perivascular Pathway of the patient in excess of a predetermined threshold; and, based on a presence of the at least one of the amyloid plaque, the Tau oligomers, or the other oligomers in the perivascular space/IPAD System/Perivascular Pathway of the patient, determining a treatment protocol for the patient, wherein the treatment protocol comprises, at least in part, administering to the patient a high density lipoprotein composition derived from mixing a blood fraction with a lipid removing agent.

Optionally, the high density lipoprotein composition is adapted to facilitate a drainage of at least one of soluble beta amyloids, soluble Tau oligomers, or other soluble oligomers.

Optionally, the method further comprises determining an amount of at least one of the amyloid plaque, the Tau oligomers, or the other oligomers in the perivascular space/IPAD System/Perivascular Pathway. Optionally, the method further comprises using diagnostic imaging to determine at least one of the presence or the extent of the at least one of the amyloid plaque, the Tau oligomers, or the other oligomers in the perivascular space of the patient.

Optionally, the drainage is facilitated via the patient’s intramural peri-arterial drainage (IPAD) pathway.

Optionally, the lipid removing agent is at least one of phenols, hydrocarbons, amines, ethers, esters, alcohols, halohydrocarbons, halocarbons, di-isopropyl ether (DIPE), diethyl ether (DEE), n-butanol, ethyl acetate, dichloromethane, chloroform, isoflurane, sevoflurane, perfluorocyclohexanes, trifluoroethane, cyclofluorohexanol, or combinations thereof.

The present specification also discloses a method for delaying the progression of, stabilizing, or improving symptoms related to cerebral amyloid angiopathy (CAA) in a patient, comprising: monitoring a pathophysiological change indicative of CAA or a potential future onset of CAA, in the patient; based on said monitoring, determining if amyloid plaque, and/or Tau oligomers, and/or other oligomers is present in a perivascular space/IPAD System/Perivascular Pathway of the patient; based on the determination of the presence of amyloid plaque, and/or Tau oligomers, and/or other oligomers in the perivascular space/IPAD System/Perivascular Pathway of the patient, determining a treatment protocol for the patient, wherein the treatment protocol comprises, at least in part, administering to the patient a high density lipoprotein composition derived from mixing a blood fraction having unmodified high density lipoproteins with a solvent to yield pre-beta high density lipoproteins, wherein the pre-beta high density lipoproteins have an increased concentration of pre-beta high density lipoprotein relative to the unmodified high density lipoproteins.

Optionally, the solvent is at least one of phenols, hydrocarbons, amines, ethers, esters, alcohols, halohydrocarbons, halocarbons, di-isopropyl ether (DIPE), diethyl ether (DEE), n-butanol, ethyl acetate, dichloromethane, chloroform, isoflurane, sevoflurane, perfluorocyclohexanes, trifluoroethane, cyclofluorohexanol, or combinations thereof.

Optionally, administering the pre-beta high density lipoproteins to the patient facilitates a drainage of soluble oligomers.

Optionally, the method further comprises: connecting the patient to a device for withdrawing blood; withdrawing blood from the patient; and separating blood cells from the blood to yield the blood fraction containing low density lipoproteins and the high density lipoproteins.

Optionally, the method further comprises delivering the high density lipoprotein composition to the patient via infusion therapy in a dosage ranging from 1 mg/kg to 250 mg/kg.

Optionally, the method further comprises delivering the high density lipoprotein composition to the patient via infusion therapy at a rate of 999 mL/hour +/- 100 mL/hr.

The present specification also discloses a method for delaying a progression of, stabilizing, or improving symptoms related to Alzheimer’s Disease (AD) in a patient, comprising: monitoring a pathophysiological change indicative of AD in a patient; based on said monitoring, determining if amyloid plaque, and/or Tau oligomers, and/or other oligomers is present in a perivascular space/IPAD System/Perivascular Pathway of the patient; determining an extent of amyloid plaque, and/or Tau oligomers, and/or other oligomers in said perivascular space/IPAD System/Perivascular Pathway ; and, based on the presence of amyloid plaque, and/or Tau oligomers, and/or other oligomers in the perivascular space/IPAD System/Perivascular Pathway of the patient, determining a treatment protocol for the patient, wherein the treatment protocol comprises administering to the patient a high density lipoprotein composition derived from mixing a blood fraction with a lipid removing agent, wherein the high density lipoprotein composition facilitates the drainage of soluble oligomers including soluble beta amyloid , Tau oligomers, and other soluble oligomers in the patient’s Intramural Peri-Arterial Drainage (IPAD) pathway.

Optionally, diagnostic imaging is used to determine the presence and extent of amyloid plaque, and/or Tau oligomers, and/or other oligomers in the perivascular space/IPAD System/Perivascular Pathway of the patient.

Optionally, the high density lipoprotein composition is derived by obtaining the blood fraction from the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the pre-beta high density lipoproteins have an increased concentration of pre-beta high density lipoproteins relative to the high density lipoproteins from the blood fraction prior to mixing.

Optionally, the pre-beta high density lipoproteins have a concentration of alpha high density lipoproteins in addition to pre-beta high density lipoproteins from the blood fraction prior to mixing.

Optionally, the pathophysiological change is indicated by an accumulation of plaque in the perivascular space/IPAD System/Perivascular Pathway of the patient resulting in cerebral amyloid angiopathy.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour +/- 100 mL/hr.

Optionally, the method further comprises determining a severity of CAA or AD in the patient using at least one of global functioning, cognitive functioning, activities of daily living, or behavioral assessments.

Optionally, after administering to the patient the high density lipoprotein composition, the patient experiences a decrease in an accumulation of the at least one of the amyloid plaque, the Tau oligomers, or other oligomers in the perivascular space/IPAD System/Perivascular Pathway.

Optionally, after administering to the patient the high density lipoprotein composition, a rate of degeneration of the patient’s physiological and/or cognitive parameters indicative of CAA decreases or indicative of AD stabilizes and does not experience a further decrease.

Optionally, after administering to the patient the high density lipoprotein composition, a rate of degeneration of the patient’s physiological and/or cognitive parameters indicative of CAA or AD, slows down relative to a rate of degeneration of the patient’s physiological and/or cognitive parameters indicative of CAA or AD before administering to the patient the high density lipoprotein composition.

Optionally, after administering to the patient the high density lipoprotein composition, the patient’s physiological and/or cognitive symptoms indicative of CAA or AD improve relative to the patient’s physiological and/or cognitive symptoms indicative of CAA or AD before administering to the patient the high density lipoprotein composition.

Optionally, the high density lipoprotein composition is derived by obtaining a blood fraction from an individual other than the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

The present specification also discloses a method for delaying the progression of, stabilizing, or improving symptoms related to Alzheimer’s Disease (AD) in a patient, comprising: monitoring a pathophysiological change indicative of AD, or a potential future onset of AD, in the patient; based on said monitoring, determining if amyloid plaque, and/or Tau oligomers, and/or other oligomers is present in a perivascular space/IPAD System/Perivascular Pathway of the patient; based on the determination of the presence of amyloid plaque, and/or Tau oligomers, and/or other oligomers in the perivascular space/IPAD System/Perivascular Pathway of the patient, determining a treatment protocol for the patient, wherein the treatment protocol comprises administering to the patient a high density lipoprotein composition derived from mixing a blood fraction, having unmodified high density lipoproteins, with a lipid removing agent to yield pre-beta high density lipoproteins, wherein the pre-beta high density lipoproteins have an increased concentration of pre-beta high density lipoprotein relative to the unmodified high density lipoproteins, and wherein the pre-beta high density lipoproteins facilitates the drainage of soluble oligomers including soluble beta amyloids, Tau oligomers, and other soluble oligomers in the patient’s Intramural Peri-Arterial Drainage (IPAD) pathway.

Optionally, the composition is derived by obtaining the blood fraction from the patient; mixing the blood fraction with the lipid removing agent to yield the pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing low density lipoproteins and the high density lipoproteins.

Optionally, the composition is derived by obtaining the blood fraction from an individual other than the patient; mixing the blood fraction with the lipid removing agent to yield the pre-beta high-density lipoproteins; separating the pre-beta high-density lipoproteins; and delivering the pre-beta high-density lipoproteins to the patient.

The present specification also discloses a method for impacting hippocampal volume indicative of Alzheimer’s Disease (AD) in a patient, comprising: determining if amyloid plaque, and/or Tau oligomers, and/or other oligomers is present in a perivascular space/IPAD System/Perivascular Pathway of the patient; determining a volume of a hippocampus of the patient; and, based on the determination of the presence of amyloid plaque, and/or Tau oligomers, and/or other oligomers in the perivascular space/IPAD System/Perivascular Pathway of the patient and the volume of the hippocampus of the patient, determining a treatment protocol for the patient, wherein the treatment protocol comprises administering to the patient a high density lipoprotein composition derived from mixing a blood fraction of the patient with a lipid removing agent.

The present specification also discloses a method for impacting hippocampal volume indicative of Alzheimer’s Disease (AD) in a person with Down syndrome, comprising: determining if amyloid plaque, and/or Tau oligomers, and/or other oligomers are present in a perivascular space/IPAD System/Perivascular Pathway of the person with Down syndrome; determining a volume of a hippocampus of the person with Down syndrome; and, based on the determination of the presence of amyloid plaque, and/or Tau oligomers, and/or other oligomers in the perivascular space/IPAD System/Perivascular Pathway of the person with Down syndrome and the volume of the hippocampus of the person with Down syndrome, determining a treatment protocol for the person with Down syndrome, wherein the treatment protocol comprises administering to the person with Down syndrome a high density lipoprotein composition derived from mixing a blood fraction of the person with Down syndrome with a lipid removing agent.

Optionally, as a therapeutic benefit, an increase in the hippocampal volume of a patient results in improved cognitive function.

The present specification also discloses a method for delaying a progression of, halting and stabilizing, or reversing and improving symptoms related to Alzheimer’s Disease (AD) in a patient, comprising: monitoring a pathophysiological change indicative of AD in a patient; based on said monitoring, determining if amyloid plaque is present in a perivascular space/IPAD System/Perivascular Pathway of the patient; determining an extent of amyloid plaque in said perivascular space/IPAD System/Perivascular Pathway ; and, based on the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction with a lipid removing agent.

Optionally, diagnostic imaging is used to determine the presence and extent of amyloid plaque in the perivascular space/IPAD System/Perivascular Pathway of the patient.

Optionally, the high density lipoprotein composition is derived by obtaining the blood fraction from the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the pre-beta high density lipoproteins have an increased concentration of pre-beta high density lipoproteins relative to the high density lipoproteins from the blood fraction prior to mixing.

Optionally, the pathophysiological change is indicated by an accumulation of plaque in the perivascular space/IPAD System/Perivascular Pathway of the patient resulting in cerebral amyloid angiopathy.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour or another rate determined best for the patient.

Optionally, the method further comprises determining a severity of AD in the patient using at least one of global functioning, cognitive functioning, activities of daily living, or behavioral assessments.

Optionally, after administering to the patient the high density lipoprotein composition, the patient experiences a halt in further accumulation or a decrease in the accumulation of amyloid plaque in the perivascular space/IPAD System/Perivascular Pathway .

Optionally, after administering to the patient the high density lipoprotein composition, a rate of degeneration of the patient’s physiological and/or cognitive parameters indicative of AD stabilizes and does not experience a further decrease.

Optionally, after administering to the patient the high density lipoprotein composition, a rate of degeneration of the patient’s physiological and/or cognitive parameters indicative of AD, slows down relative to a rate of degeneration of the patient’s physiological and/or cognitive parameters indicative of AD before administering to the patient the high density lipoprotein composition.

Optionally, after administering to the patient the high density lipoprotein composition, the patient’s physiological and/or cognitive symptoms indicative of AD improve relative to the patient’s physiological and/or cognitive symptoms indicative of AD before administering to the patient the high density lipoprotein composition.

Optionally, the high density lipoprotein composition is derived by obtaining the blood fraction from an individual other than the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

The present specification also discloses a method for delaying the progression of, halting and stabilizing, or reversing and improving symptoms related to Alzheimer’s Disease (AD) in a patient, comprising: monitoring a pathophysiological change indicative of AD, or a potential future onset of AD, in the patient; based on said monitoring, determining if amyloid plaque is present in a perivascular space/IPAD System/Perivascular Pathway of the patient; based on the determination of the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction, having unmodified high density lipoproteins, with a lipid removing agent to yield pre-beta high density lipoproteins, wherein the pre-beta high density lipoproteins have an increased concentration of pre-beta high density lipoprotein relative to the unmodified high density lipoproteins.

Optionally, the composition is derived by obtaining the blood fraction from the patient; mixing the blood fraction with the lipid removing agent to yield the pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing low density lipoproteins and the high density lipoproteins.

Optionally, the composition is derived by obtaining the blood fraction from an individual other than the patient; mixing the blood fraction with the lipid removing agent to yield the pre-beta high-density lipoproteins; separating the pre-beta high-density lipoproteins; and delivering the pre-beta high-density lipoproteins to the patient.

The present specification also discloses a method for improving an impairment of cognitive function indicative of Alzheimer’s Disease (AD) in a patient, comprising: determining if amyloid plaque is present in a perivascular space/IPAD System/Perivascular Pathway of the patient; determining an extent or severity of cognitive impairment in the patient using at least one of a global, cognitive, functional or behavioral assessment test; and, based on the determination of the presence of amyloid plaque in the perivascular space/IPAD System/Perivascular Pathway of the patient and said extent or severity of cognitive impairment in the patient, determining a treatment protocol for the patient, wherein the treatment protocol comprises administering to the patient a high density lipoprotein composition derived from mixing a blood fraction of the patient with a lipid removing agent.

Optionally, the method further comprises determining an extent of amyloid plaque in the perivascular space/IPAD System/Perivascular Pathway and determining the treatment protocol based at least in part on the determined extent of amyloid plaque.

Optionally, the high density lipoprotein composition comprises pre-beta high density lipoproteins having an increased concentration of pre-beta high density lipoprotein relative to high density lipoproteins from the blood fraction prior to mixing.

Optionally, the composition is derived by: obtaining the blood fraction from the patient; mixing said blood fraction with the lipid removing agent to yield pre-beta high-density lipoproteins; separating said pre-beta high-density lipoproteins; and delivering said pre-beta high-density lipoproteins to said patient.

Optionally, the AD is indicated by at least one of homozygous familial hypercholesterolemia, heterozygous familial hypercholesterolemia, ischemic stroke, coronary artery disease, acute coronary syndrome, or peripheral arterial disease.

Optionally, periodically monitoring changes comprises monitoring changes within a period of three to six months.

Optionally, the mixing the blood fraction with a lipid removing agent yields pre-beta high density lipoprotein that has an increased concentration of pre-beta high density lipoprotein relative to total protein.

The present specification also discloses a method for delaying a progression of, stabilizing, or improving symptoms related to Cerebral Amyloid Angiopathy (CAA) in a patient, comprising: monitoring a pathophysiological change indicative of CAA in a patient; based on said monitoring, determining if amyloid plaque is present in a perivascular space/IPAD System/Perivascular Pathway of the patient; determining an extent of amyloid plaque in said perivascular space/IPAD System/Perivascular Pathway ; and, based on the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction with a lipid removing agent.

Optionally, diagnostic imaging is used to determine the presence and extent of amyloid plaque in the perivascular space/IPAD System/Perivascular Pathway of the patient.

Optionally, the high density lipoprotein composition is derived by obtaining the blood fraction from the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the pre-beta high density lipoproteins have an increased concentration of pre-beta high density lipoproteins relative to the high density lipoproteins from the blood fraction prior to mixing.

Optionally, the pre-beta high density lipoproteins have a concentration of alpha high density lipoproteins in addition to pre-beta high density lipoproteins from the blood fraction prior to mixing.

Optionally, the pathophysiological change is indicated by an accumulation of plaque in the perivascular space/IPAD System/Perivascular Pathway of the patient resulting in CAA.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour +/- 100 mL/hr.

Optionally, after administering to the patient the high density lipoprotein composition, the patient experiences a decrease in the accumulation of amyloid plaque in the perivascular space/IPAD System/Perivascular Pathway .

Optionally, after administering to the patient the high density lipoprotein composition, a rate of degeneration of the patient’s physiological parameters indicative of CAA stabilizes and does not experience a further decrease.

Optionally, after administering to the patient the high density lipoprotein composition, a rate of degeneration of the patient’s physiological parameters indicative of CAA, slows down relative to a rate of degeneration of the patient’s physiological parameters indicative of CAA before administering to the patient the high density lipoprotein composition.

Optionally, after administering to the patient the high density lipoprotein composition, the patient’s physiological symptoms indicative of CAA improve relative to the patient’s physiological symptoms indicative of CAA before administering to the patient the high density lipoprotein composition.

Optionally, the high density lipoprotein composition is derived by obtaining the blood fraction from an individual other than the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the method further comprises delaying a progression of, stabilizing, or improving symptoms related to Cerebral Amyloid Angiopathy (CAA) in a patient, the symptoms comprising Hereditary Cerebral Amlyoid Angiopathy (HCAA).

Optionally, the method further comprises delaying a progression of, stabilizing, or improving symptoms related to Cerebral Amyloid Angiopathy (CAA) in a patient, the symptoms comprising Hereditary Cerebral Hemorrhage With Amyloidosis (HCHWA).

The present specification also discloses a method for delaying the progression of, stabilizing, or improving symptoms related to Cerebral Amyloid Angiopathy (CAA) in a patient, comprising: monitoring a pathophysiological change indicative of CAA, or a potential future onset of CAA, in the patient; based on said monitoring, determining if amyloid plaque is present in a perivascular space/IPAD System/Perivascular Pathway of the patient; based on the determination of the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction, having unmodified high density lipoproteins, with a lipid removing agent to yield pre-beta high density lipoproteins, wherein the pre-beta high density lipoproteins have an increased concentration of pre-beta high density lipoprotein relative to the unmodified high density lipoproteins.

Optionally, the composition is derived by obtaining the blood fraction from the patient; mixing the blood fraction with the lipid removing agent to yield the pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing low density lipoproteins and the high density lipoproteins.

Optionally, the composition is derived by obtaining the blood fraction from an individual other than the patient; mixing the blood fraction with the lipid removing agent to yield the pre-beta high-density lipoproteins; separating the pre-beta high-density lipoproteins; and delivering the pre-beta high-density lipoproteins to the patient.

The present specification also discloses a method for improving symptoms related to Hemorrhagic Stroke (HS) in a patient, comprising: monitoring a condition indicative of HS in a patient; based on said monitoring, determining if amyloid plaque is present in a perivascular space/IPAD System/Perivascular Pathway of the patient; determining an extent of amyloid plaque in said perivascular space/IPAD System/Perivascular Pathway; and, based on the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction with a lipid removing agent.

Optionally, diagnostic imaging is used to determine the presence and extent of amyloid plaque in the perivascular space/IPAD System/Perivascular Pathway of the patient.

Optionally, the high density lipoprotein composition is derived by obtaining the blood fraction from the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the pre-beta high density lipoproteins have an increased concentration of pre-beta high density lipoproteins relative to the high density lipoproteins from the blood fraction prior to mixing.

Optionally, the pre-beta high density lipoproteins have a concentration of alpha high density lipoproteins in addition to pre-beta high density lipoproteins from the blood fraction prior to mixing.

Optionally, the condition is indicated by an accumulation of plaque in the perivascular space/IPAD System/Perivascular Pathway of the patient resulting in HS.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour +/- 100 mL/hr.

Optionally, after administering to the patient the high density lipoprotein composition, the patient experiences a decrease in the accumulation of amyloid plaque in the perivascular space/IPAD System/Perivascular Pathway.

Optionally, after administering to the patient the high density lipoprotein composition, a rate of degeneration of the patient’s physiological parameters indicative of HS stabilizes and does not experience a further decrease.

Optionally, after administering to the patient the high density lipoprotein composition, a rate of degeneration of the patient’s physiological parameters indicative of HS, slows down relative to a rate of degeneration of the patient’s physiological parameters indicative of HS before administering to the patient the high density lipoprotein composition.

Optionally, after administering to the patient the high density lipoprotein composition, the patient’s physiological symptoms indicative of HS improve relative to the patient’s physiological symptoms indicative of HS before administering to the patient the high density lipoprotein composition.

Optionally, the high density lipoprotein composition is derived by obtaining the blood fraction from an individual other than the patient, wherein the blood fraction has high-density lipoproteins; mixing the blood fraction with the lipid removing agent to yield pre-beta high-density lipoproteins; separating the pre-beta high-density lipoproteins; and delivering the pre-beta high-density lipoproteins to the patient.

The present specification also discloses a method for improving symptoms related to Hemorrhagic Stroke (HS) in a patient, comprising: monitoring a condition indicative of HS, or a potential future onset of HS, in the patient; based on said monitoring, determining if amyloid plaque is present in a perivascular space/IPAD System/Perivascular Pathway of the patient; based on the determination of the presence of amyloid plaque in the perivascular space/IPAD System/Perivascular Pathway of the patient, determining a treatment protocol for the patient, wherein the treatment protocol comprises administering to the patient a high density lipoprotein composition derived from mixing a blood fraction, having unmodified high density lipoproteins, with a lipid removing agent to yield pre-beta high density lipoproteins, wherein the pre-beta high density lipoproteins have an increased concentration of pre-beta high density lipoprotein relative to the unmodified high density lipoproteins.

Optionally, the composition is derived by obtaining the blood fraction from the patient; mixing the blood fraction with the lipid removing agent to yield the pre-beta high-density lipoproteins; separating the pre-beta high-density lipoproteins; and delivering the pre-beta high-density lipoproteins to the patient.

Optionally, the method further comprises connecting the patient to a device for withdrawing blood; withdrawing blood from the patient; and separating blood cells from the blood to yield the blood fraction containing low density lipoproteins and the high density lipoproteins.

Optionally, the composition is derived by obtaining the blood fraction from an individual other than the patient; mixing the blood fraction with the lipid removing agent to yield the pre-beta high-density lipoproteins; separating the pre-beta high-density lipoproteins; and delivering the pre-beta high-density lipoproteins to the patient.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a flow chart delineating the steps of treating cholesterol and amyloid deposit related diseases using the treatment systems and methods in accordance with embodiments of the present specification;

FIG. 2 is a schematic representation of a plurality of components used in accordance with some embodiments of the present specification to achieve the processes disclosed herein;

FIG. 3 is a pictorial illustration of an exemplary embodiment of a configuration of a plurality of components used in accordance with some embodiments of the present specification to achieve the processes disclosed herein;

FIG. 4A is a flowchart describing a plurality of exemplary steps of a therapeutic protocol for treating a patient presenting with cerebral amyloid angiopathy (CAA), in accordance with an embodiment of the present specification;

FIG. 4B is a flowchart describing a plurality of exemplary steps of a therapeutic protocol for treating a patient presenting with cerebral amyloid angiopathy (CAA) using a CETP inhibitor, in accordance with an embodiment of the present specification;

FIG. 4C is a flowchart describing a plurality of exemplary steps of a therapeutic protocol for treating a patient presenting with cerebral amyloid angiopathy (CAA) using a CETP inhibitor and pre-beta HDL particles, in accordance with an embodiment of the present specification;

FIG. 5 is a longitudinal transverse cross-sectional view of a cerebral blood vessel illustrating removal of beta amyloid by transport along a cerebral lymphatic IPAD System/Perivascular Pathway, in accordance with an embodiment of the present specification;

FIG. 6 is a longitudinal transverse cross-sectional view of a cerebral blood vessel illustrating amyloid accumulation in a cerebral lymphatic IPAD System/Perivascular Pathway of an individual having a high level of the ε4 allele, in accordance with an embodiment of the present specification;

FIG. 7A is a longitudinal transverse cross-sectional view of a cerebral blood vessel of a patient being treated for cerebral amyloid angiopathy (CAA), in accordance with an embodiment of the present specification;

FIG. 7B illustrates a mechanism of removal of beta amyloid molecules by infusing pre-β HDL particles within the blood vessel of FIG. 7A, in accordance with an embodiment of the present specification;

FIG. 7C shows modified pre-β HDL particles flowing through the blood stream of the blood vessel of FIG. 7A, in accordance with an embodiment of the present specification;

FIG. 8A is a flowchart describing a plurality of exemplary steps of a therapeutic protocol for treating an AD patient, in accordance with an embodiment of the present specification;

FIG. 8B is a flowchart describing a plurality of exemplary steps of a therapeutic protocol for treating an AD patient using a CETP inhibitor, in accordance with an embodiment of the present specification;

FIG. 8C is a flowchart describing a plurality of exemplary steps of a therapeutic protocol for treating an AD patient using a CETP inhibitor and pre-beta HDL particles, in accordance with an embodiment of the present specification;

FIG. 9A illustrates plaque in a carotid artery of a patient, in accordance with an example;

FIG. 9B illustrates plaque in a middle cerebral artery of a patient, in accordance with an example;

FIG. 9C illustrates an embolus lodged within a central cerebral artery, in accordance with an example;

FIG. 10A is a flowchart describing a plurality of exemplary steps of a therapeutic protocol for treating a patient of hemorrhagic stroke in the presence of CAA, in accordance with an embodiment of the present specification

FIG. 10B is a flowchart describing a plurality of exemplary steps of a therapeutic protocol for treating a patient of hemorrhagic stroke in the presence of CAA using a CETP inhibitor, in accordance with an embodiment of the present specification; and

FIG. 10C is a flowchart describing a plurality of exemplary steps of a therapeutic protocol for treating a patient of hemorrhagic stroke in the presence of CAA using a CETP inhibitor and pre-beta HDL particles, in accordance with an embodiment of the present specification.

### DETAILED DESCRIPTION

The present specification relates to methods and systems for treating cholesterol-related diseases. Some embodiments of the present specification monitor changes in one or more atheroma areas and volumes in a patient, regularly over a period of time. Atheroma areas and volumes are monitored using known imaging techniques, for lipid-containing degenerative material in stenosis.

In accordance with embodiments of the present specification, based on the results of the monitoring, treatment is provided if accumulated lipid-containing degenerative material is identified to be present and above a threshold value. The treatment is repeated each time the atheroma areas and volumes are monitored, at pre-defined time intervals, and accumulated lipid-containing degenerative material is identified to be present and above the threshold.

Embodiments of the present specification treat the condition through systems, apparatuses and methods useful for removing lipid from α-High Density Lipoprotein (α-HDL) particles derived primarily from plasma of the patient thereby creating pre-beta HDL particles with reduced lipid content, particularly reduced cholesterol content. Embodiments of the present specification create these pre-beta HDL particles with reduced lipid content without substantially modifying LDL particles. Embodiments of the present specification modify original α-HDL particles to yield pre-beta HDL particles that have an increased concentration of pre-β HDL relative to the original HDL.

It has been shown that pre-β HDL dramatically increases the selective removal of cholesterol from lipid-loaded macrophages, wherein the cholesterol is associated with an increased risk of Alzheimer’s disease (AD). Pre-β HDL has also been shown to regress atherosclerosis and atheroma volumes, in addition to markers of inflammation. Additionally, pre-β HDL has a higher functional capacity than native HDL to transport proteins. Moreover, Apo A-1 (contained within pre-β HDL particles) levels are significantly lower in AD patients and are highly correlated to the severity of the AD as measured by Mini Mental State (MMSE) scores of AD patients. Using mouse model AD studies, it has been postulated that overexpression of human Apo A-1 (Preβ-HDL) reduces CAA and preserves cognitive function.

Additionally, HDL has been found to be a transport vehicle for Aβ. Native HDL has been shown to facilitate removal of soluble Aβ and attenuate CAA in a novel bioengineered human vessel model of AD. Therefore, embodiments of the present specification utilize the pre-beta HDL particles that have an increased concentration of pre-β HDL relative to the original HDL to remove Aβ for treatment of the progression of AD.

Further, the newly formed derivatives of HDL particles (pre-beta HDL) are administered to the patient to enhance cellular cholesterol efflux and treat cardiovascular diseases and/or other lipid-associated diseases, including Atheroembolic Renal Disease (AERD). The regular periodic monitoring and treatment process renders the methods and systems of the present specification more effective in treating cardiovascular diseases including Homozygous Familial Hypercholesterolemia (HoFH), Heterozygous Familial Hypercholesterolemia (HeFH), Ischemic stroke, Coronary Artery Disease (CAD), Acute Coronary Syndrome (ACS), peripheral arterial disease (PAD), Renal Arterial Stenosis (RAS), Cerebral Amyloid Angiopathy (CAA), Hereditary CAA, Hereditary cerebral hemorrhage with amyloidosis, Hemorrhagic Stroke (HS), and for treating the progression of cerebral cognitive impairment such as Alzheimer’s Disease and/or vascular dementia.

In the embodiments of the present specification, any administration protocol of the HDL composition that is disclosed with respect to one indication (i.e. Alzheimer’s disease) may be used with any of the other indications (i.e. CAA, HS) unless stated otherwise.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be openended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

The term “fluid” may be defined as fluids from animals or humans that contain lipids or lipid containing particles, fluids from culturing tissues and cells that contain lipids and fluids mixed with lipid-containing cells. For purposes of this specification, decreasing the amount of lipids in fluids includes decreasing lipids in plasma and particles contained in plasma, including but not limited to HDL particles. Fluids include, but are not limited to: biological fluids; such as blood, plasma, serum, lymphatic fluid, cerebrospinal fluid, peritoneal fluid, pleural fluid, pericardial fluid, various fluids of the reproductive system including, but not limited to, semen, ejaculatory fluids, follicular fluid and amniotic fluid; cell culture reagents such as normal sera, fetal calf serum or serum derived from any animal or human; and immunological reagents, such as various preparations of antibodies and cytokines from culturing tissues and cells, fluids mixed with lipid-containing cells, and fluids containing lipid-containing organisms, such as a saline solution containing lipid-containing organisms. A preferred fluid treated with the methods of the present specification is plasma.

The term “lipid” may be defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “extraction solvent” or “lipid removing agent” may be defined as one or more solvents used for extracting lipids from a fluid or from particles within the fluid. This solvent enters the fluid and remains in the fluid until removed by other subsystems. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1, 1,3, 3,3- hexafluoro-2- (fluoromethoxy) propane-d3), perfluorocyclohexanes, trifluoroethane, cyclofluorohexanol, and combinations thereof.

The term “patient” refers to animals and humans, which may be either a fluid source to be treated with the methods of the present specification or a recipient of derivatives of HDL particles and or plasma with reduced lipid content.

The term “HDL particles” encompasses several types of particles defined based on a variety of methods such as those that measure charge, density, size and immuno-affinity, including but not limited to electrophoretic mobility, ultracentrifugation, immunoreactivity and other methods known to one of ordinary skill in the art. Such HDL particles include but are not limited to the following: α-HDL, pre-β HDL (including pre-β1 HDL, pre-β2 HDL and pre- β3HDL), HDL2 (including HDL2a and HDL2b), HDL3, VHDL, LpA-I, LpA-II, LpA-I/LpA-II (for a review see Barrans et al. , Biochemica Biophysica Acta 1300 ; 73-85,1996). Accordingly, practice of the methods of the present specification creates pre-beta HDL particles. These modified derivatives of HDL particles may be modified in numerous ways including but not limited to changes in one or more of the following metabolic and/or physico-chemical properties (for a review see Barrans et al., Biochemica Biophysica Acta 1300; 73-85,1996); molecular mass (kDa); charge; diameter; shape; density; hydration density; flotation characteristics; content of cholesterol; content of free cholesterol; content of esterified cholesterol; molar ratio of free cholesterol to phospholipids; immuno-affinity; content, activity or helicity of one or more of the following enzymes or proteins: ApoA-I, ApoA-II, ApoD, ApoE, ApoJ, ApoA-IV, cholesterol ester transfer protein (CETP), lecithin; cholesterol acyltransferase (LCAT); capacity and/or rate for cholesterol binding, capacity and/or rate for cholesterol transport.

The term “blockage due to lipid content” is measured in a percentage and is used to refer to the extent of physical blockage in an artery.

### Lipid-Related Diseases

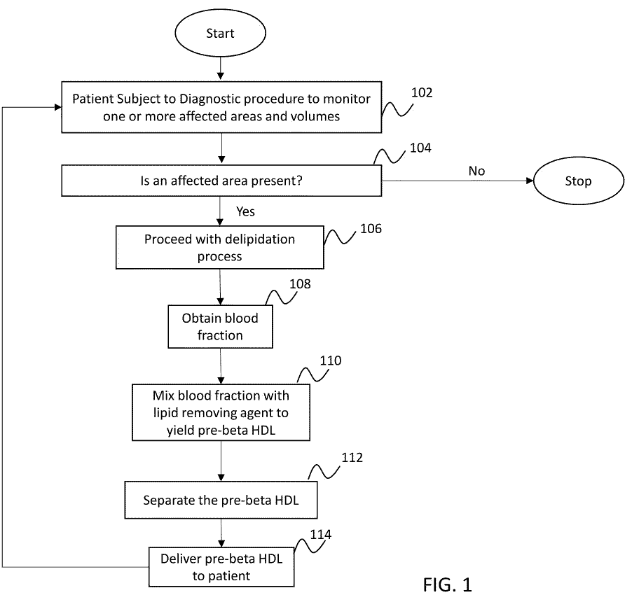

FIG. 1 is a flow chart illustrating an exemplary process of treating lipid-related diseases including cerebral diseases, such as, but not limited to stroke conditions such as CAA, Hemorrhagic stroke, Hereditary CAA, Hereditary cerebral hemorrhage with amyloidosis, and for treating the progression of cerebral cognitive impairment such as Alzheimer’s Disease in accordance with some embodiments of the present specification. At step 102, a subject or a patient who is diagnosed with a cerebral disease is monitored for one or more atheroma areas and/or volumes via a diagnostic procedure. In an embodiment, advanced medical imaging techniques, such as, but not limited to Computer Tomography (CT) angiogram and/or Intravascular Ultrasound (IVUS), may be used to detect areas within the inner layer of artery walls where lipid-containing degenerative material may have accumulated. Accumulated degenerative material may include amyloids, or fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restriction of blood flow.

If arteries with lipid-containing atheroma lesion/plaque/area/region(s) having an amount or volume of blockage within a predetermined range or as indicated by any of the diagnostic procedures outlined below are identified at 106, the patient is then subjected to the delipidation process. In this case, at step 108, a blood fraction of the patient is obtained. The process of blood fractionation is typically performed by filtration, centrifuging the blood, aspiration, or any other method known to persons skilled in the art. Blood fractionation separates the plasma from the blood. In one embodiment, blood is withdrawn from a patient in a volume sufficient to produce about 12ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, the blood fraction obtained at step 108 includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

At step 110, the blood fraction obtained at 108 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains pre-beta HDL particles with reduced lipid content. The process is designed such that HDL particles are treated to reduce their lipid levels and yield pre-beta HDL particles without destruction of plasma proteins or substantially affecting LDL particles.

Energy is introduced into the system in the form of varied mixing methods, time, and speed. At 112, bulk solvents are removed from the pre-beta HDL particles via centrifugation. In embodiments, any remaining soluble solvent is removed via charcoal adsorption, evaporation, or Hollow Fiber Contractors (HFC) pervaporation. The mixture is optionally tested for residual solvent via use of gas chromatography (GC), or similar means. The test for residual solvent may optionally be eliminated based on statistical validation.

At 114, the treated plasma containing pre-beta HDL particles with reduced lipid content, which was separated from the solvents at 112, is treated appropriately and subsequently returned to the patient. The pre-beta HDL particles are HDL particles with an increased concentration of pre-beta HDL. Concentration of pre-beta HDL is greater in the pre-beta HDL, relative to the original HDL that was present in the plasma before treating it with the solvent. Embodiments of the present specification utilize the pre-beta HDL particles that have an increased concentration of pre-β HDL relative to the original HDL to remove Aβ for treatment of progression of AD. The resulting treated plasma containing the HDL particles with reduced lipid and increased pre-beta concentration is optionally combined with the patient’s red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid or amyloid-containing degenerative material. Therefore, the process is repeated from step 102, as described above. In some embodiments, the patient is monitored repeatedly within a period of three to six months. The treatment cycle is also repeated at this frequency until the monitoring suggests substantial or complete removal of lipid or amyloid-related degenerative material that cause cerebral diseases. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

In an optional embodiment, which may be implemented in at least one treatment protocol as described in the present specification, a CETP inhibitor is used to increase HDL levels in a patient. In an optional embodiment, which may be implemented in at least one treatment protocol as described in the present specification, a CETP inhibitor is used in conjunction with the delipidation process described throughout the specification. More specifically, a CETP inhibitor is in a class of compounds that inhibit cholesteryl ester transfer protein (CETP), which normally transfers cholesterol from HDL cholesterol to very low density or low density lipoproteins (VLDL or LDL). Inhibition of this process results in higher HDL levels and reduces LDL levels. Thus, in embodiments, the CETP inhibitor is used to increase the plasma level of HDL. The HDL (wherein the increased levels are created by the delipidation process which generates pre-beta HDL and/or by the use of a CETP inhibitor) will bind to the amyloid that may be present in plasma and/or the perivascular space/IPAD System/Perivascular Pathway. When bound to one or both of pre-beta HDL generated by the delipidation process or CETP-inhibited HDL, the amyloid may be transported (now in increased levels) to a site of degradation, thereby decreasing levels of amyloid. In embodiments, the degradation site is the liver. In embodiments, the CETP inhibitor binds to amyloid beta peptides in the peripheral vasculature of the patient, before the amyloid beta peptides have crossed the blood brain barrier and into the brain. In some embodiments, a CETP inhibitor is administered to the patient without administering pre-beta HDL particles, resulting in a decrease in flux of amyloid beta peptides into the brain. In other embodiments, a CETP inhibitor in administered to the patient in conjunction with the administration of pre-beta HDL particles, resulting in a decrease in flux of amyloid beta peptides into the brain (as peripheral amyloid beta peptides are bound by the CETP inhibitor and transported to a degradation site) and an increase in the removal of amyloid beta peptides from the brain (as amyloid beta peptide in the brain is bound by the pre-beta HDL particles, removed from the brain, and transported to a degradation site).

In embodiments, the delipidation process of the present specification may be used in a short-term therapeutic approach (boosts), or intermittently, while the use of a CETP inhibitor may be used as a chronic, regular therapeutic approach. In embodiments, a combination therapy comprises the use of a CETP inhibitor as a chronic, regular therapeutic application with an intermittent application of pre-beta HDL particles.

Embodiments of the present specification create a natural, functional pre-β HDL, which contain ApoA-I. Normally, pre-β HDL comprises only 5% of the total HDL in circulation. Embodiments of the present specification describe processes that dramatically increase this ratio to over 80% of the total HDL in circulation. It has been observed that pre-β HDL derived in accordance with the disclosed methods reduce plaques in coronary arteries of patients with heart disease to a three times greater extent in about seven short weeks than is seen in 2.5 years for statin therapy. In addition, pre-β HDL substantially reduces inflammation. Furthermore, pre-β HDL is more efficient than native HDL in removing Aβ for treatment of progression of AD.

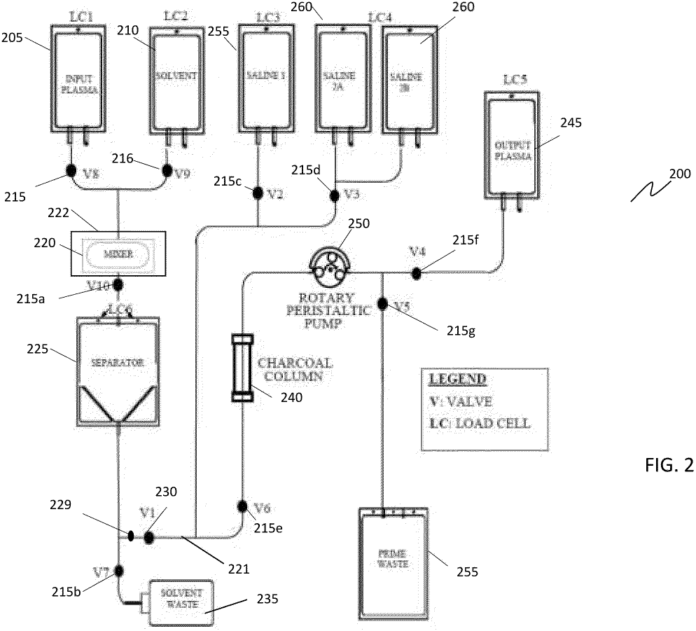

FIG. 2 illustrates an exemplary embodiment of a system and its components used to achieve the methods of the present specification. The figure depicts an exemplary basic component flow diagram defining elements of the HDL modification system 200. Embodiments of the components of system 200 are utilized after obtaining a blood fraction from a patient or another individual (donor). The plasma, separated from the blood is brought in a sterile bag to system 200 for further processing. The plasma may be separated from blood using a known plasmapheresis device. The plasma may be collected from the patient into a sterile bag using standard apheresis techniques. The plasma is then brought in the form of a fluid input to system 200 for further processing. In embodiments, system 200 is not connected to the patient at any time and is a discrete, stand-alone system for delipidating plasma and creating pre-beta HDL particles. The patient’s plasma is processed by system 200 and brought back to the patient’s location to be reinfused back into the patient. In alternate embodiments, the system may be a continuous flow system that is connected to the patient in which both plasmapheresis and delipidation are performed in an excorporeal, parallel system and the delipidated plasma product is returned to the patient.

A fluid input 205 (containing blood plasma) is provided and connected via tubing to a mixing device 220. A solvent input 210 is provided and also connected via tubing to mixing device 220. In embodiments, valves 215, 216 are used to control the flow of fluid from fluid input 205 and solvent from solvent input 210 respectively. It should be appreciated that the fluid input 205 contains any fluid that includes HDL particles, including plasma having LDL particles or devoid of LDL particles, as discussed above. It should further be appreciated that solvent input 210 can include a single solvent, a mixture of solvents, or a plurality of different solvents that are mixed at the point of solvent input 210. While depicted as a single solvent container, solvent input 210 can comprise a plurality of separate solvent containers. Embodiments of types of solvents that may be used are discussed above.

Mixer 220 mixes fluid from fluid input 205 and solvent from solvent input 210 to yield a fluid-solvent mixture. In embodiments, mixer 220 is capable of using a shaker bag mixing method with the input fluid and input solvent in a plurality of batches, such as 1, 2, 3 or more batches. An exemplary mixer is a Barnstead Labline orbital shaker table. In alternative embodiments, other known methods of mixing are utilized. In some embodiments, mixer 220 includes shaker table 222. Once formed, the fluid-solvent mixture is directed, through tubing and controlled by at least one valve 215a, to a separator 225. In an embodiment, separator 225 is capable of performing bulk solvent separation through gravity separation in a funnel-shaped bag.

In separator 225, the fluid-solvent mixture separates into a first layer and second layer. The first layer comprises a mixture of solvent and lipid that has been removed from the HDL particles. The first layer is transported through a valve 215b to a first waste container 235. The second layer comprises a mixture of residual solvent, pre-beta HDL particles, and other elements of the input fluid. One of ordinary skill in the art would appreciate that the composition of the first layer and the second layer would differ based upon the nature of the input fluid. Once the first and second layers separate in separator 225, the second layer is transported through tubing to a solvent extraction device 240. In an embodiment, a pressure sensor 229 and valve 230 is positioned in the flow stream to control the flow of the second layer to solvent extraction device 240.

The opening and closing of valves 215, 216 to enable the flow of fluid from input containers 205, 210 may be timed using mass balance calculations derived from weight determinations of the fluid inputs 205, 210 and separator 225. For example, the valve 215b between separator 225 and first waste container 235 and valve 230 between separator 225 and solvent extraction device 240 open after the input masses (fluid and solvent) substantially balances with the mass in separator 225 and a sufficient period of time has elapsed to permit separation between the first and second layers. Depending on what solvent is used, and therefore which layer settles to the bottom of separator 225, either valve 215b between separator 225 and first waste container 235 is opened or valve 230 between separator 225 and solvent extraction device 240 is opened. One of ordinary skill in the art would appreciate that the timing of the opening is dependent upon how much fluid is in the first and second layers and would further appreciate that it is preferred to keep valve 215b between separator 225 and first waste container 235 open just long enough to remove all of the first layer and some of the second layer, thereby ensuring that as much solvent as possible has been removed from the fluid being sent to solvent extraction device 240.

In embodiments, an infusion grade fluid (“IGF”) may be employed via one or more inputs 260 which are in fluid communication with the fluid path 221 leading from separator 225 to solvent extraction device 240 for priming. In an embodiment, saline is employed as the infusion grade priming fluid in at least one of inputs 260. In an embodiment, 0.9% sodium chloride (saline) is employed. In other embodiments, glucose may be employed as the infusion grade priming fluid in any one of inputs 260.

In embodiments, a glucose input 255 and one or more saline inputs 260 are in fluid communication with the fluid path 221 leading from separator 225 to solvent extraction device 240. A plurality of valves 215c and 215d are also incorporated in the flow stream from glucose input 255 and saline input 260 respectively, to the tubing providing the flow path 221 from separator 225 to solvent extraction device 240. IGF such as saline and/or glucose are incorporated into embodiments of the present specification in order to prime solvent extraction device 240 prior to operation of the system. In embodiments, saline is used to prime most of the fluid communication lines and solvent extraction device 240. If priming is not required, the IGF inputs are not employed. Where such priming is not required, the glucose and saline inputs are not required. Also, one of ordinary skill in the art would appreciate that the glucose and saline inputs can be replaced with other primers if required by the solvent extraction device 240.

In some embodiments, solvent extraction device 240 is a charcoal column designed to remove the specific solvent used in solvent input 210. An exemplary solvent extraction device 240 is an Asahi Hemosorber charcoal column, or the Bazter/Gambro Adsorba 300C charcoal column or any other charcoal column that is employed in blood hemoglobin perfusion procedures. A pump 250 is used to move the second layer from separator 225, through solvent extraction device 240, and to an output container 245. In embodiments, pump 250 is a rotary peristaltic pump, such as a Masterflex Model 77201-62.

The first layer is directed to waste container 235 that is in fluid communication with separator 225 through tubing and at least one valve 215b. Additionally, other waste, if generated, can be directed from the fluid path connecting solvent extraction device 240 and output container 245 to a second waste container 255. Optionally, in an embodiment, a valve 215f is included in the path from the solvent extraction device 240 to the output container 245. Optionally, in an embodiment, a valve 215g is included in the path from the solvent extraction device 240 to the second waste container 255.

In an embodiment of the present specification, gravity is used, wherever practical, to move fluid through each of the plurality of components. For example, gravity is used to drain input plasma 205 and input solvent 210 into mixer 220. Where mixer 220 comprises a shaker bag and separator 225 comprises a funnel bag, fluid is moved from the shaker bag to the funnel bag and, subsequently, to first waste container 235, if appropriate, using gravity.

In an additional embodiment, not shown in FIG. 2, the output fluid in output container 245 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. The output fluid is returned to the bloodstream of the patient and the solvent concentrations must be below a predetermined level to carry out this operation safely. In embodiments, the sensors are capable of providing such concentration information on a real-time basis and without having to physically transport a sample of the output fluid, or air in the headspace, to a remote device. The resultant separated pre-beta HDL particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

Referring back to FIG. 2, suitable materials for use in any of the apparatus components as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present specification, during at least a single use. The materials are sterilizable either by radiation or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers L.L.C., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 215, 215a, 215b, 215c, 215d, 215e, 215f, 215g, 216 and any other valve used in each embodiment may be composed of, but are not limited to, pinch, globe, ball, gate or other conventional valves. In some embodiments, the valves are occlusion valves such as Acro Associates’ Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

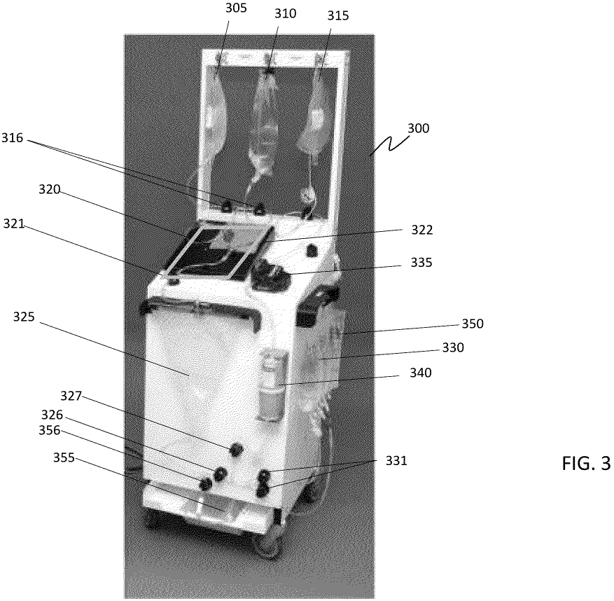

FIG. 3 illustrates an exemplary configuration of a system used in accordance with some embodiments of the present specification to achieve the processes disclosed herein. Referring to FIG. 3, a configuration of basic components of the HDL modification system 300 is shown. A fluid input 305 is provided and connected via tubing to a mixing device 320. A solvent input 310 is provided and also connected via tubing to a mixing device 320. Preferably valves 316 are used to control the flow of fluid from fluid input 305 and solvent from solvent input 310. It should be appreciated that the fluid input 305 preferably contains any fluid that includes HDL particles, including plasma having LDL particles or devoid of LDL particles, as discussed above. It should further be appreciated that solvent input 310 can include a single solvent, a mixture of solvents, or a plurality of different solvents that are mixed at the point of solvent input 310. While depicted as a single solvent container, solvent input 310 can comprise a plurality of separate solvent containers. The types of solvents that are used and preferred are discussed above.

The mixer 320 mixes fluid from fluid input 305 and solvent from solvent input 310 to yield a fluid-solvent mixture. Preferably, mixer 320 is capable of using a shaker bag mixing method with the input fluid and input solvent in a plurality of batches, such as 1, 2, 3 or more batches. In some embodiments, mixer 320 includes shaker table 322. Once formed, the fluid-solvent mixture is directed, through tubing and controlled by at least one valve 321, to a separator 325. In a preferred embodiment, separator 325 is capable of performing bulk solvent separation through gravity separation in a funnel-shaped bag.

In the separator 325, the fluid-solvent mixture separates into a first layer and second layer. The first layer comprises a mixture of solvent and lipid that has been removed from the HDL particles. The second layer comprises a mixture of residual solvent, pre-beta HDL particles, and other elements of the input fluid. One of ordinary skill in the art would appreciate that the composition of the first layer and the second layer would differ based upon the nature of the input fluid. Once the first and second layers separate in separator 325, the second layer is transported through tubing to a solvent extraction device 340. Preferably, a pressure sensor 326 and valve 327 is positioned in the flow stream to control the flow of the second layer to the solvent extraction device 340.

Preferably, a glucose input 330 and saline input 350 is in fluid communication with the fluid path leading from the separator 325 to the solvent extraction device 340. A plurality of valves 331 is also preferably incorporated in the flow stream from the glucose input 330 and saline input 350 to the tubing providing the flow path from the separator 325 to the solvent extraction device 340. Glucose and saline are incorporated into the present specification in order to prime the solvent extraction device 340 prior to operation of the system. Where such priming is not required, the glucose and saline inputs are not required. Also, one of ordinary skill in the art would appreciate that the glucose and saline inputs can be replaced with other primers if the solvent extraction device 340 requires it.

The solvent extraction device 340 is preferably a charcoal column designed to remove the specific solvent used in the solvent input 310. An exemplary solvent extraction device 340 is an Asahi Hemosorber charcoal column. A pump 335 is used to move the second layer from the separator 325, through the solvent extraction device 340, and to an output container 315. The pump is preferably a peristaltic pump, such as a Masterflex Model 77201-62.

The first layer is directed to a waste container 355 that is in fluid communication with separator 325 through tubing and at least one valve 356. Additionally, other waste, if generated, can be directed from the fluid path connecting solvent extraction device 340 and output container 315 to waste container 355.

Preferably, an embodiment of the present specification uses gravity, wherever practical, to move fluid through each of the plurality of components. For example, preferably gravity is used to drain the input plasma 305 and input solvent 310 into the mixer 320. Where the mixer 320 comprises a shaker bag and separator 325 comprises a funnel bag, fluid is moved from the shaker bag to the funnel bag and, subsequently, to the waste container 355, if appropriate, using gravity.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

In embodiments, the delipidation systems illustrated in FIGS. 2 and 3, including the shaker tables 222 and 322, are configured to reduce a time required to complete a delipidation process, relative to delipidation systems of the prior art, by a range of 1% to 50%. In embodiments, the delipidation systems illustrated in FIGS. 2 and 3, including the shaker tables 222 and 322, are configured to increase a percentage of delipidation of alpha HDL to pre-beta HDL relative to delipidation systems of the prior art. In some embodiments, the delipidation systems illustrated in FIGS. 2 and 3, including the shaker tables 222 and 322, are configured to increase a percentage of delipidation of alpha HDL to pre-beta HDL up to at least 90%, relative to delipidation systems of the prior art which are configured to provide a percentage of delipidation of alpha HDL to pre-beta HDL of approximately 66%.

To enable the operation of the above described embodiments of the present specification, it is preferable to supply a user of such embodiments with a packaged set of components, in kit form, comprising each component required to practice embodiments of the present specification. The kit may include an input fluid container (i.e. a high density lipoprotein source container), a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

### Cerebral Amyloid Angiopathy (CAA)

Cerebral Amyloid Angiopathy (CAA) is an aging-related condition caused by deposits of amyloid proteins in the wall or perivascular space, intramural peri-arterial drainage (IPAD) system, or perivascular pathway of blood vessels in a brain. The perivascular space comprises fluid-filled structures in the brain around the blood vessels, the perivascular pathway is a waste clearance system in the brain, also not in the blood vessels, and the intramural peri-arterial drainage (IPAD) system is a drainage pathway along the basement membranes in the capillaries and arteries of the blood vessels. Low levels of CAA may usually be harmless, however, severe CAA may lead to the protein deposits causing the blood vessels to crack, in which case the blood can leak out and damage the brain. Amyloids are similar to the deposits in the brain that cause Alzheimer’s disease (AD). Amyloid peptides may be produced in the brain and deposit there, causing disease, and may also be produced peripherally, outside of the brain, cross the blood brain barrier, and become trapped and deposit in the brain, causing disease. The causes known to increase risks of CAA include advancing age, accompanying presence of AD, and some type of genes. Specifically, the gene known as Apolipoprotein E is considered to be a risk factor for CAA. CAA is also estimated to be the cause of 30-40% of hemorrhagic strokes. Differential diagnosis may be performed to determine the probability of CAA in a patient. Imaging tests like CT scans or MRI scans can show whether a bleeding occurred in the outer part of the brain (the cortex) where CAA is usually most severe. This can help distinguish CAA from hemorrhagic strokes caused by high blood pressure, which tend to occur in deep sections of the brain. In addition, a kind of MRI scan called gradient-echo MRI can show whether there have been other tiny areas of bleeding that are also in the typical locations for CAA.

A Modified Boston Criteria incorporates cortical superficial siderosis into the radiological diagnosis to determine a probability of CAA. The criteria comprises of combined clinical, imaging and pathological parameters. The criteria has four tiers:
o Tier 1 represents definite CAA, and determined during a full post-mortem examination. The examination reveals lobar, cortical, or cortical/subcortical hemorrhage and pathological evidence of severe CAA.
o Tier 2 represents probable CAA with supporting pathological evidence. This examination may not be post-mortem. Clinical data and pathological tissue (evacuated hematoma or cortical biopsy specimen) demonstrate a hemorrhage as mentioned above, and some degree of vascular amyloid deposition, indicative of CAA.
o Tier 3 represents probable CAA. In this case pathological confirmation is not required. Patients of 55 years or older with an appropriate clinical history are considered. Additionally, MRI findings demonstrate multiple hemorrhages restricted to lobar, cortical, or corticosubcortical regions (cerebellar hemorrhages allowed) of varying sizes/ages without another cause. Alternatively, a single lobar, cortical, or corticosubcortical hemorrhage and focal (three or less sulci) or disseminated (more than three sulci) cortical superficial siderosis without another cause.
o Tier 4 represents possible CAA. This is also applicable to patients of 55 years or older age with an appropriate clinical history. Additionally, MRI findings demonstrate a single, (more than three sulci) cortical superficial siderosis without another; or focal or disseminated cortical superficial siderosis without another cause.

Apolipoprotein E (ApoE) is a class of proteins involved in the metabolism of fats in the body and is the principal cholesterol carrier in the brain. ApoE is polymorphic, with three major alleles, namely ApoE-ε2, ApoE-ε3, and ApoE-ε4. ApoE-ε2 has an allele frequency of approximately 7% to 8% in the general population. This variant of the apolipoprotein binds poorly to cell surface receptors while ApoE-ε3 and ApoE-ε4 bind relatively well. ApoE-ε2 is associated with both increased and decreased risk for atherosclerosis. Individuals with a ε2/ε2 combination tend to clear dietary fat more slowly and be at greater risk for early vascular disease and the genetic disorder type III hyperlipoproteinemia. ApoE-ε3 has an allele frequency of approximately 80% in the general population. It is considered the “neutral” ApoE genotype of the three. ApoE-ε4 has an allele frequency of approximately 14% in the general population. Beta amyloid (Aβ) particles are accumulated in the cerebral IPAD System/Perivascular Pathway due to an increased presence of ApoE-ε4 particles. As a result, beta amyloid is deposited in the walls of the blood vessel as CAA.

In aspects of the present specification, a treated plasma that contains pre-beta HDL particles with reduced lipid content is delivered to the patient via infusion therapy. The process is designed such that HDL particles are treated to reduce their lipid levels and yield pre-beta HDL particles without destruction of plasma proteins or substantially affecting LDL particles.

The HDL lipoprotein particles are comprised of ApoA-I, phospholipids and cholesterol. Persons of ordinary skill in the art would appreciate that Apolipoprotein A-I (ApoA-I) particles comprise of two sub-fractions, pre-β HDL and α-HDL, which have pre-beta and alpha electrophoretic mobility, respectively. Thus, pre-β HDL represents ApoA-I molecules complexed with phospholipids.

In aspects of the present specification, a treated plasma that contains pre-beta HDL particles with reduced lipid content is delivered to the patient via infusion therapy. In an embodiment, the pre-beta high density lipoproteins have a concentration of alpha high density lipoproteins in addition to the pre-beta high density lipoproteins from the blood fraction prior to mixing.

In aspects of the present specification, isolated pre-β HDL particles are infused into the patient’s blood stream to bind to beta amyloid particles and clear the cerebral IPAD System/Perivascular Pathway.

Referring back to FIG. 1, the process of removing beta amyloid particles and clearing the cerebral is explained from the step of diagnosing a patient with a cerebral disease to the step of delivering pre-beta HDL to the patient.

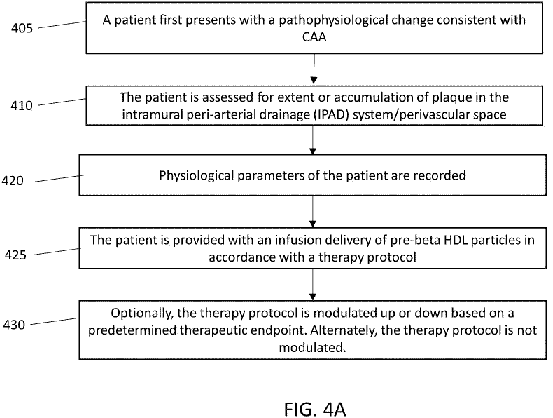

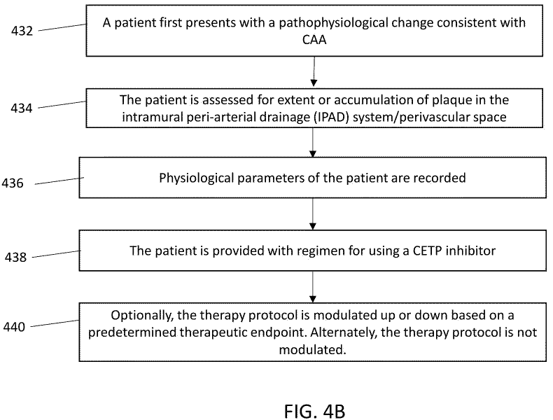

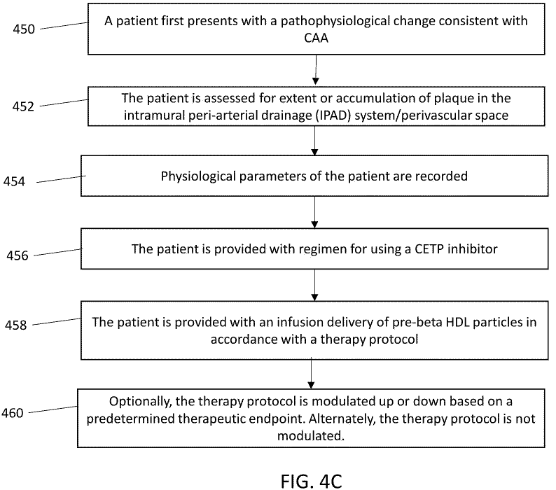

FIG. 4A is a flowchart describing a plurality of exemplary steps of a therapy protocol for treating a CAA patient, in accordance with an embodiment of the present specification. At step 405, a patient first presents with a pathophysiological change that is consistent with symptoms of CAA. Any of the aforementioned diagnostic techniques may be used in this step. In embodiments various biomarkers may be used to determine the pathophysiological change. For example, measuring the level of amyloids may be used to assess the extent of a pathophysiological change characteristic of CAA. At step 410, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET) and Magnetic Resonance Imaging (MRI), may be used.

At step 420, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 405.

At step 425, the patient is infused with pre-beta HDL particles in accordance with a therapy protocol. In an optional embodiment, a blood fraction is withdrawn from the patient presenting with the amyloid deposits. In an alternative embodiment, a blood fraction is obtained withdrawing blood from a person other than the patient. Therefore, the plasma obtained as a result of the blood fractionation process may be either autologous or non-autologous. The blood fraction is subsequently treated, using the delipidation process described above in context of FIG. 1, to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100 ml/hour or a rate deemed more appropriate for the patient. In embodiments, the treatment is performed once, or may be repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

At optional step 430, the therapy protocol may be titrated or modulated up or down based on a therapeutic endpoint.

FIG. 4B is a flowchart describing a plurality of exemplary steps of a therapeutic protocol for treating a patient presenting with cerebral amyloid angiopathy (CAA) using a CETP inhibitor, in accordance with an embodiment of the present specification. At step 432, a patient first presents with a pathophysiological change that is consistent with symptoms of CAA. Any of the aforementioned diagnostic techniques may be used in this step. In embodiments various biomarkers may be used to determine the pathophysiological change. For example, measuring the level of amyloids may be used to assess the extent of a pathophysiological change characteristic of CAA. At step 434, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET) and Magnetic Resonance Imaging (MRI), may be used.

At step 436, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 432.

At step 438, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

At optional step 440, the therapy protocol may be titrated or modulated up or down based on a therapeutic endpoint.

In an optional embodiment, which may be implemented in at least one treatment protocol as described in the present specification, a CETP inhibitor is used in conjunction with the delipidation process described throughout the specification. In embodiments, the delipidation process of the present specification may be used in a short-term therapeutic approach (boosts), or intermittently, while the use of a CETP inhibitor may be used as a chronic, regular therapeutic approach. It should be noted that the steps shown FIG. 4A and FIG. 4B may be combined for an embodiment in which the CETP inhibitor is used in conjunction with the delipidation process. In embodiments, a combination therapy comprises the use of a CETP inhibitor as a chronic, regular therapeutic application with an intermittent application of pre-beta HDL particles.

FIG. 4C is a flowchart describing a plurality of exemplary steps of a therapeutic protocol for treating a patient presenting with cerebral amyloid angiopathy (CAA) using a CETP inhibitor and pre-beta HDL particles, in accordance with an embodiment of the present specification. At step 450, a patient first presents with a pathophysiological change that is consistent with symptoms of CAA. Any of the aforementioned diagnostic techniques may be used in this step. In embodiments various biomarkers may be used to determine the pathophysiological change. For example, measuring the level of amyloids may be used to assess the extent of a pathophysiological change characteristic of CAA. At step 452, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET) and Magnetic Resonance Imaging (MRI), may be used.

At step 454, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 450.

At step 456, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

It should be noted that in various embodiments of the present specification, administration of a CETP inhibitor is provided on a chronic basis as described above.

At step 458, the patient is infused with pre-beta HDL particles in accordance with a therapy protocol. In an optional embodiment, a blood fraction is withdrawn from the patient presenting with CAA. In an alternative embodiment, a blood fraction is obtained withdrawing blood from a person other than the patient. Therefore, the plasma obtained as a result of the blood fractionation process may be either autologous or non-autologous.

The blood fraction is subsequently treated, using the delipidation process described above to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

It should be noted that in various embodiments of the present specification, administration of pre-beta HDL particles, as described in step 458, is performed on an intermittent basis during chronic administration of a CETP inhibitor, as described in step 456.

At optional step 460, the therapy protocol may be titrated or modulated up or down based on a therapeutic endpoint.

### Therapeutic Protocols for Administering Pre-Beta HDL Particles for CAA

In embodiments, one or more intra-treatment severity level assessments are made using diagnostic and/or physical procedures/tests. The one or more intra-treatment severity level assessments are made at predetermined points in time during the course of therapy. If the intra-treatment severity level assessments show a delay in the onset of additional symptoms, a halting in the worsening of symptoms, or an improvement in the patient’s condition, it is considered to be of therapeutic benefit. In embodiments, when therapeutic benefit is shown, the therapeutic amount may be titrated down wherein parameters such as, but not limited to, the dose range, frequency or cycle of treatment and/or course of therapy may be reduced. Alternately, the therapy protocol may be titrated up depending on various factors. Still alternately, if the intra-treatment severity level assessments show or do not show improvement in the patient’s condition, the therapy protocol is not modulated.

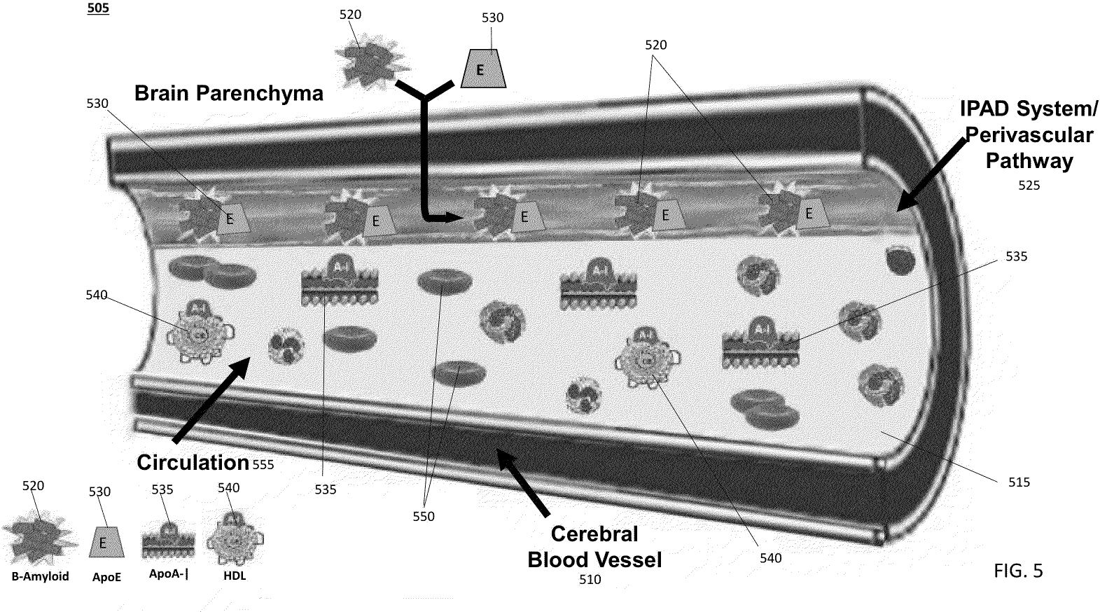

FIG. 5 is a longitudinal transverse cross-sectional view 505 of a cerebral blood vessel 510 illustrating removal of beta amyloid by transport along the cerebral lymphatic IPAD System/Perivascular Pathway, in accordance with an embodiment of the present specification. As shown, blood circulates through the lumen 515 of the vessel 510 while Interstitial Fluid (ISF) and solutes, including beta amyloid (Aβ) 520, are eliminated from the brain through the perivascular drainage pathway 525, which is, effective, the lymphatic drainage of the brain. The ε3 allele 530 binds to beta amyloid particles 520, forming modified ε3 particles, and thereby transporting beta amyloid particles 520 from the brain along the perivascular drainage pathway 525. Also shown are Apolipoprotein A-I (ApoA-I) particles 535 and HDL particles 540 as part of the blood circulation 555 through the lumen 515 along with other particles such as, for example, red blood cells 550.

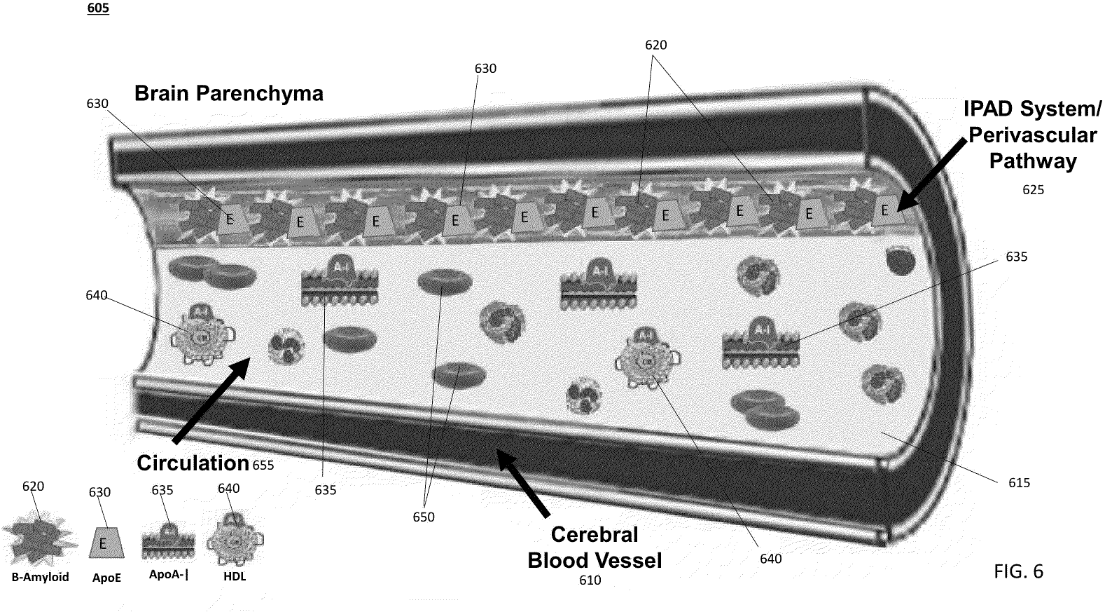

FIG. 6 is a longitudinal transverse cross-sectional view 605 of a cerebral blood vessel 610 illustrating amyloid accumulation in cerebral lymphatic perivascular pathways of individuals with an increased presence of the ε4 allele, in accordance with an embodiment of the present specification. As shown, blood circulates through the lumen 615 of the vessel 610 while beta amyloid (Aβ) particles 620 are accumulated in the cerebral IPAD System/Perivascular Pathway 625 due to an increased presence of ε4 particles 630. Thus, beta amyloid 620 is deposited in the walls of the blood vessel 610 as CAA. CAA reflects a failure of elimination of amyloid-beta (Aβ) from the brain along perivascular lymphatic drainage pathways 625. Failure of elimination of beta amyloid along perivascular pathways may coincide with a reduction in enzymatic degradation of beta amyloid, reduced absorption of beta amyloid into the blood and stiffening of blood vessel walls. Also shown are ApoA-I particles 635 and HDL particles 640 as part of the blood circulation 655 through the lumen 615 along with other particles such as, for example, red blood cells 650.

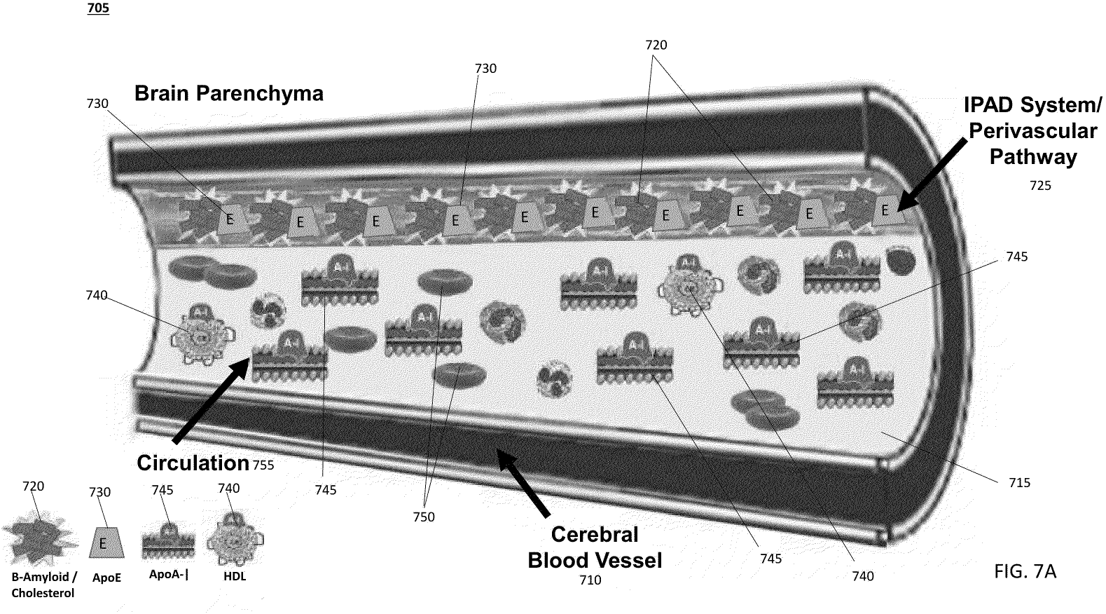

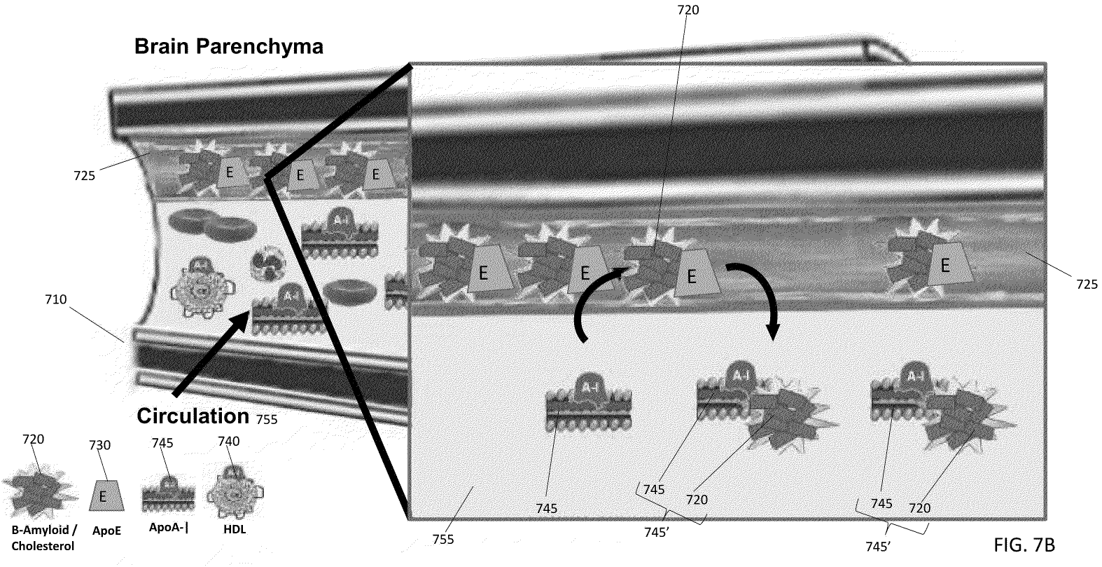

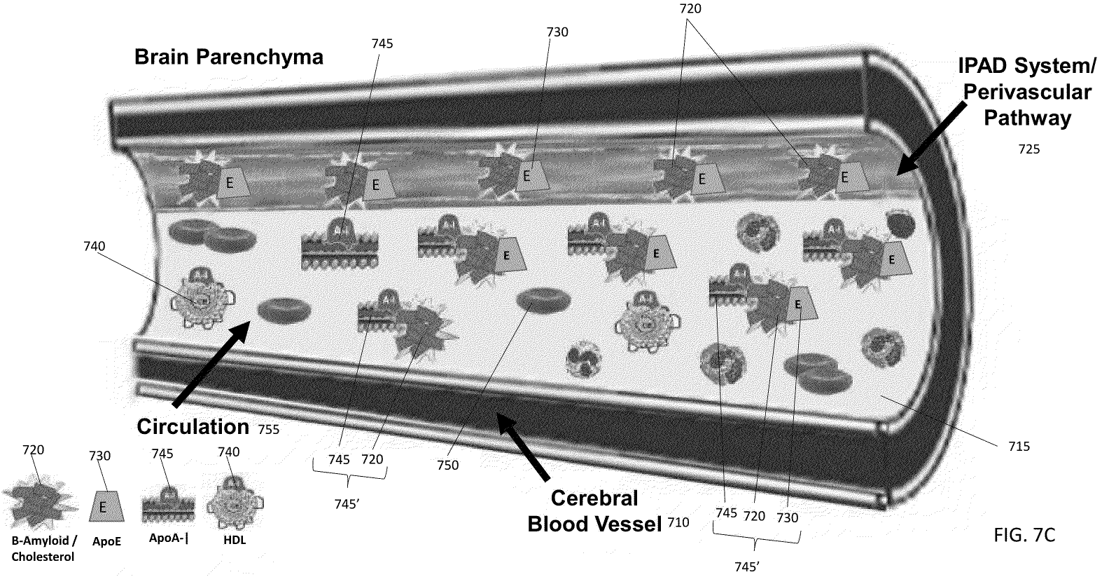

FIG. 7A is a longitudinal transverse cross-sectional view 705 of a cerebral blood vessel 710 of a patient being treated for cerebral amyloid angiopathy (CAA), in accordance with an embodiment of the present specification. As shown, blood circulates through the lumen 715 of the vessel 710 while beta amyloid (Aβ) particles 720 accumulate in the cerebral IPAD System/Perivascular Pathway 725 along with a high presence of ε4 particles 730, thereby essentially blocking pathway 725. In accordance with an aspect of the present specification, treated plasma or isolated pre-β HDL particles 745 are infused into the patient’s blood stream 755 to bind to beta amyloid particles 720 and clear the cerebral IPAD System/Perivascular Pathway 725. Pre-β HDL 745 represents ApoA-I molecules complexed with phospholipids.

To generate and subsequently infuse the patient with treated plasma or with a solution containing an increased concentration of isolated pre-β HDL 745, a blood fraction is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

In some alternate embodiments, Low Density Lipoprotein (LDL) is also separated from the plasma. Separated LDL is usually discarded. In alternative embodiments, LDL is retained in the plasma. In accordance with embodiments of the present specification, the resultant blood fraction includes plasma with HDL, and may or may not include other protein particles.

In one embodiment, the process of blood fractionation is performed by withdrawing blood from the patient, and who is being treated by the physician. In an alternative embodiment, the process of blood fractionation is performed by withdrawing blood from a person other than the patient. Therefore, the plasma obtained as a result of the blood fractionation process may be either autologous or non-autologous.

In an optional embodiment, the autologous or non-autologous plasma obtained is subjected to a delipidation process as described in greater detail above with respect to FIG. 1 but repeated briefly herein. The resultant blood fraction is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. The plasma and solvent are introduced into at least one apparatus for mixing, agitating, or otherwise contacting the plasma with the solvent. The plasma may be transported using a continuous or batch process. The solvents dissolve lipids from the plasma. In embodiments of the present specification, the solvents dissolve lipids to yield treated plasma that contains pre-beta HDL particles with reduced lipid content. The process is designed such that HDL particles are treated to reduce their lipid levels and yield pre-beta HDL particles without destruction of plasma proteins or substantially affecting LDL particles. The resultant treated plasma containing pre-beta HDL particles with reduced lipid content, which was separated from the solvents, is treated appropriately and may be subsequently returned to the patient in an embodiment.

In an optional embodiment, the resultant fluid containing pre-beta HDL particles is further processed, in a second stage, to separate or to isolate pre-β HDL particles. In an embodiment, the second stage occurs in a separate and discrete area from the delipidation process. In an alternate embodiment, the second stage processing occurs in-line with the delipidation system, whereby the system may be connected to an affinity column sub-system or ultracentrifugation sub-system. The resultant separated pre-β HDL particles may then be introduced to the bloodstream of the patient as described below.

Referring back to FIG. 7A, a presence of non-modified HDL particles 740 is illustrated in the blood stream 755 along with other particles such as, for example, red blood cells 750. The pre-beta HDL particles may be HDL particles with an increased concentration of pre-β HDL particles 745. Concentration of pre-β HDL 745 is greater in the pre-beta HDL, relative to the original HDL that was present in the plasma before treating it with the solvent. The resulting treated plasma containing the HDL particles with reduced lipid and increased pre-β concentration is optionally combined with the patient’s red blood cells, if the red cells were not already returned during plasmapheresis, and administered to the patient. One route of administration is through the vascular system, preferably intravenously, such as via infusion therapy.

FIG. 7B illustrates a mechanism of removal of beta amyloid molecules 720 by infused pre-β HDL particles 745 within the blood vessel 710 of a patient, in accordance with an embodiment of the present specification. As shown, with increased concentration of pre-β HDL particles 745 in the patient’s blood stream 755, a relatively higher number of pre-β HDL particles 745 are available to bind to and pull out beta-amyloid particles 720 from the IPAD System/Perivascular Pathway 725. The pre-β HDL particles 745 in the blood stream 755 enter the IPAD System/Perivascular Pathway 725 and bind with beta-amyloid particles 720 to form modified pre-β HDL particles 745′ that re-enter the blood stream 755.

FIG. 7C shows a plurality of modified pre-β HDL particles 745′ flowing in the blood stream 755 (in the lumen 715 of the blood vessel 710) and serving to transport the bound beta amyloid 720 to the liver for degradation and subsequent excretion. Thus, the infused isolated pre-β HDL particles 745 initiate reverse cholesterol, specifically beta amyloid 720, transport process from the cerebral perivascular pathways 725 to liver. Also seen in FIG. 7C are non-modified HDL particles 740 in the blood stream 755 along with other particles such as, for example, red blood cells 750.

### Alzheimer’s Disease (AD) and CAA

Alzheimer’s disease is determined using results from several tests to arrive at a differential diagnosis. Thus, there is no definitive diagnosis for Alzheimer’s disease. In embodiments, treatments and protocols of the present specification are applicable to patients exhibiting pre-symptomatology of AD, in addition to symptoms related to altered global function, cognitive function, activities of daily living (ADL)/functional impairment, and behavior. Thus, patients suffering from AD can be characterized as having early stage (pre-symptomatic)/Stages 1-4, mild, moderate, or severe AD based upon the totality of symptoms.

The following categories may be assigned and are provided for the design and evaluation of benefits throughout the different early stages of AD:
o Stage 1 is representative of a class of patients with characteristic pathophysiologic changes of early onset AD but no evidence of clinical impact. These patients are truly asymptomatic with no subjective complaint, functional impairment, or detectable abnormalities on sensitive neuropsychological measures. The characteristic pathophysiologic changes are typically demonstrated by assessment of various biomarker measures.
o Stage 2 includes the group of patients with characteristic pathophysiologic changes of early onset AD and subtle detectable abnormalities on sensitive neuropsychological measures, but no functional impairment. The emergence of subtle functional impairment signals a transition to Stage 3.
o Stage 3 is representative of a class of patients with characteristic pathophysiologic changes of early onset AD, subtle or more apparent detectable abnormalities on sensitive neuropsychological measures, and mild but detectable functional impairment. The functional impairment in this stage is not severe enough to warrant a diagnosis of overt dementia.
o Stage 4 includes a group of patients with overt dementia. This diagnosis is made as functional impairment worsens from that seen in Stage 3. This stage may be refined into additional categories which correspond to mild, moderate, and severe Alzheimer’s disease states as described below.
o Stages 5, 6, and 7 correspond to increasing degrees of overt dementia and/or cerebral functional impairment. As such, stages 5, 6, and 7 correspond to mild, moderate, and severe AD.

In embodiments, a baseline, starting or initial severity level is diagnosed/assessed using at least one physiological diagnostic or advanced medical imaging technique. In some embodiments, a baseline, starting or initial severity level is additionally assessed by at least one cognitive measurement or test. Given the panoply of available neuropsychological tests, a pattern of putatively beneficial effects demonstrated across multiple individual tests may be used to assess impact in early AD or a large magnitude of effect on a single sensitive measure of neuropsychological performance may be used. For example, measuring the level of amyloid peptide (including 40 and 42) may be used to assess a possible treatment benefit.

Differential diagnosis and the assessment of the severity level of Alzheimer’s disease may be based on one or more global, cognitive, functional and behavioral measurements, assessments, or tests.

In embodiments, global assessment tests may include assessments such as, but not limited to Clinician’s Interview-Based Impression of Change plus caregiver assessment (the CIBIC-plus), and Clinical Dementia Rating-sum of boxes (CDR-SB).

Clinician’s Interview-Based Impression of Change plus caregiver input (the CIBIC-plus) is not a single or standardized instrument, such as the ADAS-cog described below. Clinical trials for investigational drugs have used a variety of CIBIC formats, each different in terms of depth and structure. As such, results from a CIBIC-plus reflect clinical experiences from the trial or trials in which it was used and cannot be compared directly with the results of CIBIC-plus evaluations from other clinical trials. By way of example, the CIBIC-plus used in some major trials is a semi-structured instrument that was intended to examine four major areas of patient function: General, Cognitive, Behavioral, and Activities of Daily Living. It represents the assessment of a skilled clinician based upon his/her observations at an interview with the patient, in combination with information supplied by a caregiver familiar with the behavior of the patient over the interval rated. The CIBIC- plus is scored as a seven-point categorical rating, ranging from a score of 1, indicating “markedly improved,” to a score of 4, indicating “no change” to a score of 7, indicating “markedly worse.” The CIBIC-plus has not been systematically compared directly to assessments not using information from caregivers (CIBIC) or other global methods.

Clinical Dementia Rating-sum of boxes (CDR-SB) measures cognitive performance in six areas: memory, orientation, judgment/problem solving, community affairs, home/hobbies, personal care. Each category is scored on five-point scale of impairment (0=none, 0.5=questionable, 1=mild, 2=moderate, 3=severe). The sum of ratings (0-18) provides the overall CDR-SB assessment.

In embodiments, cognitive tests may include assessments such as, but not limited to, the cognitive subscale of the Alzheimer’s disease Assessment Scale (ADAS-cog) and Mini Mental State Examination (MMSE).

The cognitive subscale of the Alzheimer’s disease Assessment Scale (ADAS-cog) is a multi-factor instrument that has been extensively validated in longitudinal cohorts of Alzheimer’s disease patients. The ADAS-cog examines selected aspects of cognitive performance including elements of memory, orientation, attention, reasoning, language, and praxis. The ADAS-cog scoring range is from 0 to 70, with higher scores indicating greater cognitive impairment. Elderly adults with normal cognitive functionality may score as low as 0 or 1, but it is not unusual for adults not presenting with typical dementia to score slightly higher.

The Mini Mental State Examination (MMSE) includes 11 questions regarding orientation, memory, concentration, language, and praxis. The scoring scale ranges from 0 to 30, with a higher score indicating lower impairment. Typically, healthy individuals score approximately 29-30 points on the MMSE. A patient with Alzheimer’s disease may typically score in the range of 20-22 points on the MMSE.

In embodiments, functional tests or tests that assess impairment in activities of daily living, may include assessments such as, but not limited to, Severe Impairment Battery (SIB), Modified Alzheimer’s disease Cooperative Study-activities of daily living inventory (ADCS-ADL) and Modified Alzheimer’s disease Cooperative Study-activities of daily living inventory for severe Alzheimer’s disease (ADCS-ADL-severe), Progressive Deterioration Scale (PDS), Instrumental Activities of Daily Living (IADL), and the Katz Activities of Daily Living (ADL) index.

The Severe Impairment Battery (SIB) assessment is a multi-item instrument and has been validated for the evaluation of cognitive function in patients presenting with moderate to severe dementia. The SIB evaluates selective aspects of cognitive performance, including elements of memory, language, orientation, attention, praxis, visuospatial ability, construction, and social interaction. The SIB scoring range is from 0 to 100, with lower scores indicating greater cognitive impairment.

The Modified Alzheimer’s Disease Cooperative Study-Activities of Daily Living inventory (ADCS-ADL) consists of a comprehensive battery of ADL questions used to measure the functional capabilities of patients. Each ADL item is rated from the highest level of independent performance to complete loss. The investigator performs the inventory by interviewing a caregiver familiar with the behavior of the patient. A subset of 19 items, including ratings of the patient’s ability to eat, dress, bathe, telephone, travel, shop, and perform other household chores has been validated for the assessment of patients with moderate to severe dementia. The modified ADCS-ADL has a scoring range of 0 to 54, with the lower scores indicative of greater functional impairment.

The Modified Alzheimer’s Disease Cooperative Study - Activities of Daily Living Inventory for Severe Alzheimer’s Disease (ADCS-ADL-severe) is derived from the Alzheimer’s disease Cooperative Study-Activities of Daily Living Inventory described above, which is a comprehensive battery of ADL questions used to measure the functional capabilities of patients. Each ADL item is rated from the highest level of independent performance to complete loss. The ADCS-ADL-severe is a subset of 19 items, including ratings of the patient’s ability to eat, dress, bathe, use the telephone, get around (or travel), and perform other activities of daily living; it has been validated for the assessment of patients with moderate to severe dementia. The ADCS-ADL-severe has a scoring range of 0 to 54, with the lower scores indicative of greater functional impairment. The investigator performs the inventory by interviewing a caregiver, such as a nurse staff member, who is familiar with the overall functional capability of the patient.

The Progressive Deterioration Scale (PDS) examines activities of daily living (ADL) and instrumental ADL in 11 areas, including the extent to which the patient can leave the immediate neighborhood, the use of familiar household implements, involvement in family finances and budgeting, self-care, and routine tasks. The scoring scale ranges from 0 to 100, wherein a higher score indicating better overall functional capability.

The Instrumental Activities of Daily Living (IADL) assessment is used to measure competence in complex ADL, including telephoning, shopping, food preparation, housekeeping, laundering, use of transportation, use of medicine, and the ability to handle money. Each behavioral area is scored 1 or 0. A higher composite score indicates better functional performance.

The Katz Activities of Daily Living (ADL) index is used to assess a patient’s ability to perform ADL independently in six functions of bathing, dressing, toileting, transferring, continence, and feeding. Each function is assigned a score of yes or no for independence in that function, whereby each “yes” answer generates one point. A total score of 6 indicates full functional capability while a score of 2 or less is indicative of severe functional impairment.

In embodiments, behavioral and mood tests may include assessments such as, but not limited to, Neuropsychiatric Inventory (NPI) and are employed to determine an extent of depression, anxiety, irritability, and overall mood shifts.

The Neuropsychiatric Inventory (NPI) evaluates 10 items including delusions, hallucinations, dysphoria, anxiety, agitation, euphoria, apathy, irritability, disinhibition, aberrant motor behavior (pacing and rummaging). Two more items may also be assessed, specifically, nighttime behavior and changes in appetite and eating behaviors. The frequency of behavioral disturbances are rated on a four-point scale with the severity rated on three-point scale. A higher total score is indicative of more behavioral problems.

In some cases, diagnostic imaging tests are used to determine the accumulation or regional lesions of plaque in the perivascular space/IPAD System/Perivascular Pathway. The advanced medical imaging techniques are used to both determine the extent of plaque in the perivascular space/IPAD System/Perivascular Pathway and to assess a severity level of Alzheimer’s disease. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

A specific Amyloid Positron Emission Tomography (PET) Scan, also referred to as Amyloid PET imaging, represents a potential major advance in an early diagnosis of Alzheimer’s disease and/or an assessment of the degree of cerebral cognitive impairment. The scan visualizes plaque regions or lesions present in the brain, which are prime suspects in damaging and killing nerve cells in Alzheimer’s patients. The scan technique employs radioactive tracers to highlight amyloid protein plaque regions or lesions within the brain, which are a hallmark of Alzheimer’s disease. Amyloid PET scanning enables the “illumination” of amyloid plaques on a brain PET scan, enabling accurate detection of plaques in living people. The scan may allow for an earlier diagnosis or assessment of Alzheimer’s disease, prior to the presentation of symptomatology.

The practice parameters for the diagnosis and evaluation of dementia, as published by the American Academy of Neurology (AAN), consider structural brain imaging optimal, wherein MRI is one of the appropriate imaging methods. The AAN suggests that neuroimaging may be most useful in patients with dementia characterized by an early onset or an unusual course. Thus, Magnetic Resonance Imaging (MRI) may be considered a preferred neuroimaging examination for diagnosis and assessment of Alzheimer’s disease because it allows for accurate measurement of the 3-dimensional (3 D) volume of brain structures, and in particular, the size of the hippocampus and related regions. HDL levels are inversely correlated with the development of AD in epidemiology studies. Elderly patients with higher levels of HDL tend to have a higher hippocampal volume; conversely, a lower hippocampal volume has been used as an index of AD disease progression. In addition, people with Down syndrome, and more specifically as exhibited in children, exhibit a lower hippocampal volume and a higher rate of developing AD than those that do not have Down syndrome. Neuroimaging is widely believed to be generally useful for excluding reversible causes of dementia syndrome, such as normal-pressure hydrocephalus, brain tumors, and subdural hematoma, and for excluding other likely causes of dementia, such as cerebrovascular disease, thereby enabling a differential diagnosis of AD.

Spinal Fluid Test (detection of Beta Amyloid Fragments), is a diagnostic test that requires drawing fluid from the spinal region. Researchers have identified a protein “signature” in the spinal fluid of patients with Alzheimer’s disease, which could represent an important advance in its diagnosis. The signature was found in the cerebrospinal fluid (CSF) of 90% of people with a diagnosis of Alzheimer’s disease and 72% of people with mild cognitive impairment (MCI) - a disorder that often progresses to Alzheimer’s. Researchers measured concentrations of three proteins previously identified as potential biological indicators, or biomarkers, for Alzheimer’s and MCI: amyloid-beta, tau, and phospho-tau. Alzheimer’s disease was identified in three independent study groups wherein the participants exhibited low levels of the amyloid protein amyloid-beta 1-42, along with high levels of total tau and elevated phospho-tau 181 (P-tau 181).

Apolipoprotein E (ApoE) is a class of proteins involved in the metabolism of fats in the body and is the principal cholesterol carrier in the brain. ApoE is polymorphic, with three major alleles, namely ApoE-ε2, ApoE-ε3, and ApoE-ε4. ApoE-ε2 has an allele frequency of approximately 7% to 8% in the general population. This variant of the apolipoprotein binds poorly to cell surface receptors while ApoE-ε3 and ApoE-ε4 bind relatively well. ApoE-ε2 is associated with both increased and decreased risk for atherosclerosis. Individuals with a ε2/ε2 combination tend to clear dietary fat more slowly and be at greater risk for early vascular disease and the genetic disorder type III hyperlipoproteinemia. ApoE-ε3 has an allele frequency of approximately 80% in the general population. It is considered the “neutral” ApoE genotype of the three. ApoE-ε4 has an allele frequency of approximately 14% in the general population. The ε4 variant is the largest known genetic risk factor for late-onset sporadic Alzheimer’s disease (AD).

Although 40-65% of AD patients have at least one copy of the ε4 allele, ApoE-ε4 is not a definitive determinant of the disease; at least one-third of patients with AD are ApoE-ε4 negative and some people with ApoE-ε4 homozygotes never develop the disease. Yet, studies show that those with two ε4 alleles have up to 20 times the risk of developing AD and thus, it can be implicated as at least a contributing factor. There is also evidence that the ApoE-ε2 allele may serve a protective role in AD. Thus, the genotype most at risk for Alzheimer’s disease and at an earlier age is ApoE-ε4, ApoE-ε4. Using genotype ApoE-ε3, ApoE-ε3 as a benchmark (allocating a risk factor of 1.0 to the persons who have this genotype), individuals with genotype ApoE-ε4, ApoE-ε4 have a relative risk factor of 14.9 of developing Alzheimer’s disease. Individuals with the ApoE-ε3, ApoE-ε4 genotype exhibit a relative risk factor of 3.2, while people with the ε2 allele and the ε4 allele (ApoE-ε2, ApoE-ε4) have a relative risk factor of 2.6. Persons with one copy each of the ε2 allele and the ε3 allele (ApoE-ε2, ApoE-ε3) have a relative risk factor of 0.6, as do persons with two copies of the 2 allele (ApoE-ε2, ApoE-ε2).

While ApoE-ε4 has been found to greatly increase the likelihood that an individual will develop Alzheimer’s disease, it should be noted that persons with any combination of independent risk factors, such as but not limited to different levels of certain ApoE alleles as described above, high overall serum total cholesterol levels, and high blood pressure have an amplified risk of developing AD at some point in their lifetime. Accordingly, research has suggested that lowering serum cholesterol levels may reduce a person’s risk for Alzheimer’s disease, even if they have two ApoE-ε4 alleles, thus reducing the risk from nine or ten times the odds of developing AD down to just two times the odds. Women are more likely to develop AD than men across most ages and persons with at least one ε4 allele have significantly more neurological dysfunction than men.

In aspects of the present specification, a treated plasma that contains pre-beta HDL particles with reduced lipid content is delivered to the patient via infusion therapy. The process is designed such that HDL particles are treated to reduce their lipid levels and yield pre-beta HDL particles without destruction of plasma proteins or substantially affecting LDL particles.

The HDL lipoprotein particles are comprised of ApoA-I, phospholipids and cholesterol. Persons of ordinary skill in the art would appreciate that Apolipoprotein A-I (ApoA-I) particles comprise of two sub-fractions, pre-β HDL and α-HDL, which have pre-beta and alpha electrophoretic mobility, respectively. Thus, pre-β HDL represents ApoA-I molecules complexed with phospholipids.

As stated above, it has been shown that pre-β HDL dramatically increases the selective removal of cholesterol from lipid-loaded macrophages, wherein the cholesterol is associated with an increased risk of Alzheimer’s disease (AD). Pre-β HDL has also been shown to regress atherosclerosis and atheroma volumes, in addition to markers of inflammation. Additionally, pre-β HDL has a higher functional capacity than native HDL to transport proteins. Moreover, Apo A-1 (contained within pre-β HDL particles) levels are significantly lower in AD patients and are highly correlated to the severity of the AD as measured by Mini Mental State (MMSE) scores of AD patients. Using mouse model AD studies, it has been postulated that overexpression of human Apo A-1 (Preβ-HDL) reduces CAA and preserves cognitive function.

Additionally, HDL has been found to be a transport vehicle for Aβ. Native HDL has been shown to facilitate removal of soluble Aβ and attenuate CAA in a novel bioengineered human vessel model of AD. Therefore, embodiments of the present specification utilize the pre-beta HDL particles that have an increased concentration of pre-β HDL relative to the original HDL to remove Aβ for treatment of the progression of AD.

In aspects of the present specification, a treated plasma that contains pre-beta HDL particles with reduced lipid content is delivered to the patient via infusion therapy. In an embodiment, the pre-beta high density lipoproteins have a concentration of alpha high density lipoproteins in addition to the pre-beta high density lipoproteins from the blood fraction prior to mixing.

In aspects of the present specification, isolated pre-β HDL particles are infused into the patient’s blood stream to bind to (Aβ), Tau oligomers, and other soluble oligomer particles and clear the cerebral IPAD System/Perivascular Pathway.

Referring back to FIG. 1, the process of removing beta amyloid (Aβ), Tau oligomers, and other soluble oligomer particles and clearing the cerebral IPAD System/Perivascular Pathway is explained from the step of diagnosing a patient with a cerebral disease to the step of delivering pre-beta HDL to the patient.

Referring again to FIG. 5, a longitudinal transverse cross-sectional view 505 of a cerebral blood vessel 510 illustrating removal of beta amyloid by transport along the cerebral lymphatic IPAD System/Perivascular Pathway is shown, in accordance with an embodiment of the present specification. As shown, blood circulates through the lumen 515 of the vessel 510 while Interstitial Fluid (ISF) and solutes, including beta amyloid (Aβ) 520, are eliminated from the brain through the perivascular drainage pathway 525, which is, effective, the lymphatic drainage of the brain. The ε3 allele 530 binds to beta amyloid particles 520, forming modified ε3 particles, and thereby transporting beta amyloid particles 520 from the brain along the perivascular drainage pathway 525. Also shown are Apolipoprotein A-I (ApoA-I) particles 535 and HDL particles 540 as part of the blood circulation 555 through the lumen 515 along with other particles such as, for example, red blood cells 550. AD is, in some cases, characterized by build-ups of aggregates of the peptide beta-amyloid in the cerebral lymphatic perivascular pathways. As illustrated in Table A, in AD patients the distribution of ε2, ε3 and ε4 alleles is approximately 4%, 60% and 37%, respectively. The isoform ApoE-ε4 is not as effective as the alleles at promoting clearance of beta amyloid from the cerebral perivascular drainage pathways. Thus, a skewed abundance of ε4 allele is associated with increased vulnerability to AD in individuals with that gene variation and in AD patients is also associated with an increase in the severity of AD and loss of cognitive function.

Referring back to FIG. 6, a longitudinal transverse cross-sectional view 605 of a cerebral blood vessel 610 illustrating amyloid accumulation in cerebral lymphatic perivascular pathways of individuals with an increased presence of the ε4 allele is illustrated, in accordance with an embodiment of the present specification. As shown, blood circulates through the lumen 615 of the vessel 610 while beta amyloid (Aβ) particles 620 are accumulated in the cerebral IPAD System/Perivascular Pathway 625 due to an increased presence of ε4 particles 630. Thus, beta amyloid 620 is deposited in the walls of the blood vessel 610 as cerebral amyloid angiopathy (CAA). CAA in AD reflects a failure of elimination of amyloid-beta (Aβ) from the brain along perivascular lymphatic drainage pathways 625. Failure of elimination of beta amyloid along perivascular pathways may coincide with a reduction in enzymatic degradation of beta amyloid, reduced absorption of beta amyloid into the blood and stiffening of blood vessel walls. Also shown are ApoA-I particles 635 and HDL particles 640 as part of the blood circulation 655 through the lumen 615 along with other particles such as, for example, red blood cells 650.

Referring again to FIG. 7A, a longitudinal transverse cross-sectional view 705 of a cerebral blood vessel 710 of an AD patient being treated for cerebral amyloid angiopathy (CAA) is shown, in accordance with an embodiment of the present specification. As shown, blood circulates through the lumen 715 of the vessel 710 while beta amyloid (Aβ) particles 720 accumulate in the cerebral IPAD System/Perivascular Pathway 725 along with a high presence of ε4 particles 730, thereby essentially blocking pathway 725. In accordance with an aspect of the present specification, treated plasma or isolated pre-β HDL particles 745 are infused into the patient’s blood stream 755 to bind to beta amyloid particles 720 and clear the cerebral IPAD System/Perivascular Pathway 725. Pre-β HDL 745 represents ApoA-I molecules complexed with phospholipids.

To generate and subsequently infuse the patient with treated plasma or with a solution containing an increased concentration of isolated pre-β HDL 745, a blood fraction is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

In some alternate embodiments, Low Density Lipoprotein (LDL) is also separated from the plasma. Separated LDL is usually discarded. In alternative embodiments, LDL is retained in the plasma. In accordance with embodiments of the present specification, the resultant blood fraction includes plasma with HDL, and may or may not include other protein particles.

In one embodiment, the process of blood fractionation is performed by withdrawing blood from the patient presenting with AD, and who is being treated by the physician. In an alternative embodiment, the process of blood fractionation is performed by withdrawing blood from a person other than the patient. Therefore, the plasma obtained as a result of the blood fractionation process may be either autologous or non-autologous.

In an optional embodiment, the autologous or non-autologous plasma obtained is subjected to a delipidation process as described in greater detail above with respect to FIG. 1 but repeated briefly herein. The resultant blood fraction is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. The plasma and solvent are introduced into at least one apparatus for mixing, agitating, or otherwise contacting the plasma with the solvent. The plasma may be transported using a continuous or batch process. The solvents dissolve lipids from the plasma. In embodiments of the present specification, the solvents dissolve lipids to yield treated plasma that contains pre-beta HDL particles with reduced lipid content. The process is designed such that HDL particles are treated to reduce their lipid levels and yield pre-beta HDL particles without destruction of plasma proteins or substantially affecting LDL particles. The resultant treated plasma containing pre-beta HDL particles with reduced lipid content, which was separated from the solvents, is treated appropriately and may subsequently be returned to the patient in an embodiment.

In an optional embodiment, the resultant fluid containing pre-beta HDL particles is further processed, in a second stage, to separate or to isolate pre-β HDL particles. In an embodiment, the second stage occurs in a separate and discrete area from the delipidation process. In an alternate embodiment, the second stage processing occurs in-line with the delipidation system, whereby the system may be connected to an affinity column sub-system or ultracentrifugation sub-system. The resultant separated pre-β HDL particles may then be introduced to the bloodstream of the patient as described below.

FIG. 7A illustrates the presence of non-modified HDL particles 740 in the blood stream 755 along with other particles such as, for example, red blood cells 750. The pre-beta HDL particles are HDL particles with an increased concentration of pre-β HDL particles 745. Concentration of pre-β HDL 745 is greater in the pre-beta HDL, relative to the original HDL that was present in the plasma before treating it with the solvent. The resulting treated plasma containing the HDL particles with reduced lipid and increased pre-β concentration is optionally combined with the patient’s red blood cells, if the red cells were not already returned during plasmapheresis, and administered to the patient. One route of administration is through the vascular system, preferably intravenously, such as via infusion therapy.

Referring again to FIG. 7B, the mechanism of removal of beta amyloid molecules 720 by infused pre-β HDL particles 745 within the blood vessel 710 of an AD patient is illustrated, in accordance with an embodiment of the present specification. As shown, with increased concentration of pre-β HDL particles 745 in the patient’s blood stream 755, a relatively higher number of pre-β HDL particles 745 are available to bind to and pull out beta-amyloid particles 720 from the IPAD System/Perivascular Pathway 725. The pre-β HDL particles 745 in the blood stream 755 enter the IPAD System/Perivascular Pathway 725 and bind with beta-amyloid particles 720 to form modified pre-β HDL particles 745′ that re-enter the blood stream 755.

Referring again to FIG. 7C, a plurality of modified pre-β HDL particles 745′ flowing in the blood stream 755 (in the lumen 715 of the blood vessel 710) and serving to transport the bound beta amyloid 720 to the liver for degradation and subsequent excretion is shown. In some embodiments, the pre-β HDL particles 745 also pull the ε4 particles 730 along with the beta amyloid molecules 720 from the IPAD System/Perivascular Pathway 725. In such embodiments, the modified pre-β HDL particles 745′ are pre-β HDL 745 binding both beta amyloid 720 and ε4 730. Thus, the infused isolated pre-β HDL particles 745 initiate reverse cholesterol, specifically beta amyloid 720, transport process from the cerebral perivascular pathways 725 to liver. Also seen in FIG. 7C are non-modified HDL particles 740 in the blood stream 755 along with other particles such as, for example, red blood cells 750.

### Therapeutic Protocols for Administering Pre-Beta HDL Particles for Alzheimer’s Disease

In accordance with aspects of the present specification, treated plasma containing pre-beta HDL particles with reduced lipid and/or increased pre-β concentration is administered to a patient in accordance with a plurality of therapeutic protocols. In some embodiments, therapy is based on a level of severity of AD, as described above. In various embodiments, the plurality of therapy protocols comprises at least one or any combination of a plurality of therapeutic parameters such as, but not limited to:

Dosing range: 1 mg/kg to 250 mg/kg, and any increment therein, where a specific fixed dose may be calculated based on one or both of a patient’s weight and the severity of the disease state.

Dosing volume: the average dosing volume is dependent upon the dose (in mg/kg) and the concentration of the product to be infused into the patient (treated plasma containing pre-beta HDL particles or isolated pre-beta particles). In embodiments, the volume that is returned to the patient is substantially equal to the volume that was removed from the patient prior to the delipidation process. In embodiments, the volume that is returned to the patient is a concentrated volume. In embodiments, the volume delivered to a patient via infusion therapy is dependent upon the preparation of the product, whether it is treated plasma or concentrated, isolated pre-beta and the overall solubility of that product in a buffer or saline.

Dosing rate: the dose is provided via infusion therapy. It should be noted herein that the rate of infusion is the normal infusion rate for intravenous therapy, or 999 mL/hour and is thus dependent on overall volume and concentration. In an embodiment, the time of infusion ranges from one hour to eight hours.

Frequency or cycle of treatment: daily, weekly, monthly and annually

Duration or course of therapy: at least one day to at least one year

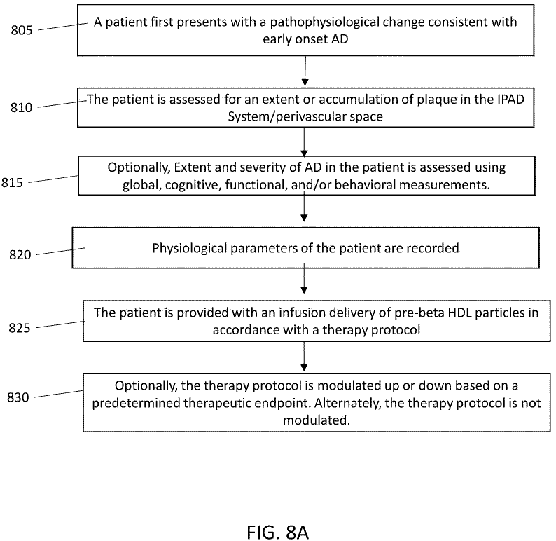

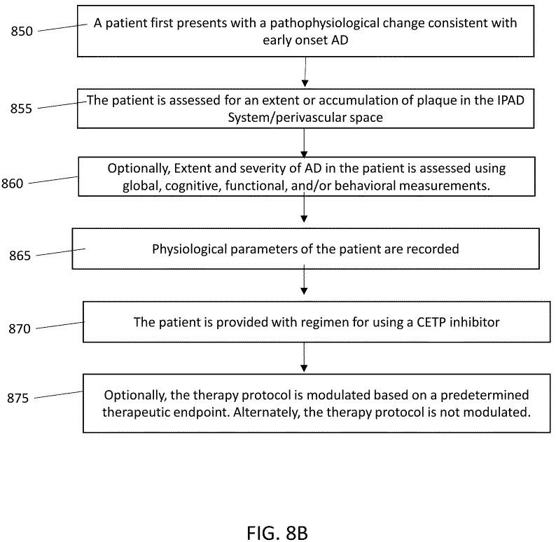

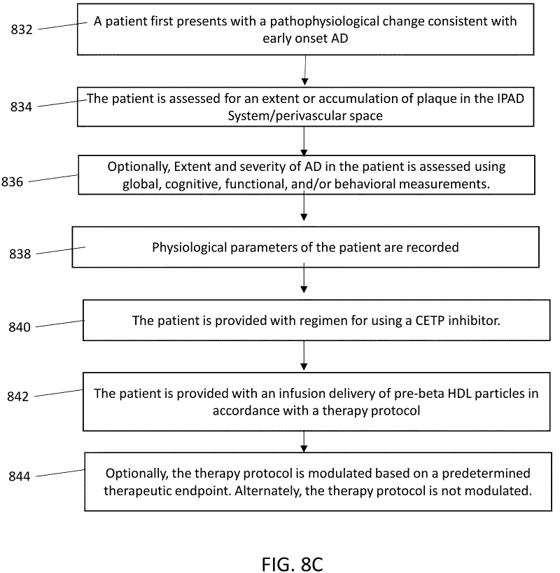

FIG. 8A is a flowchart describing a plurality of exemplary steps of a therapy protocol for treating an AD patient, in accordance with an embodiment of the present specification. At step 805, a patient first presents with a pathophysiological change that is consistent with early onset AD. Any of the aforementioned diagnostic techniques may be used in this step. In embodiments various biomarkers may be used to determine the pathophysiological change. For example, measuring the level of amyloid peptide (including 40 and 42) may be used to assess the extent of a pathophysiological change characteristic of AD. In embodiments, the ratio of beta amyloid peptide 42 to beta amyloid peptide 40 is measured to determine and assess the extent of a pathophysiological change characteristic of AD and to assess the efficacy of treatment. Particularly, the ratio of beta amyloid peptide 42 to beta amyloid peptide 40 in plasma and in cerebrospinal fluid is reduced in patients with AD. An AD patient may present with a ratio of beta amyloid peptide 42 to beta amyloid peptide 40 of approximately 0.057, while an individual without AD may have a ratio of 0.073. In an embodiment, the patient may present with cerebral amyloid angiopathy (CAA) as detected using imaging and/or biomarker techniques. For example, in embodiments, CAA patients could present with microbleeds and/or dilated perivascular space/IPAD System/Perivascular Pathways upon imaging and/or biomarker studies relative to individuals without CAA. A change in the incidence in microbleeds and/or size of the perivascular space/IPAD System/Perivascular Pathway may be used to assess efficacy of treatment. At step 810, a patient who is diagnosed with AD is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

Spinal Fluid Test (detection of Beta Amyloid Fragments), is a diagnostic test that requires drawing fluid from the spinal region. Researchers have identified a protein “signature” in the spinal fluid of patients with CAA, which could represent an important advance in its diagnosis. The signature was found in the cerebrospinal fluid (CSF) of 90% of people with a diagnosis of Alzheimer’s disease and 72% of people with mild cognitive impairment (MCI) - a disorder that often progresses to Alzheimer’s. Researchers measured concentrations of three proteins previously identified as potential biological indicators, or biomarkers, for Alzheimer’s and MCI: amyloid-beta, tau, and phospho-tau. Alzheimer’s disease was identified in three independent study groups wherein the participants exhibited low levels of the amyloid protein amyloid-beta 1-42, along with high levels of total tau and elevated phospho-tau 181 (P-tau 181).

In embodiments, at optional step 815, the diagnosis and severity level of AD in the patient are additionally assessed based on one or more global, cognitive, functional, and behavioral measurements or tests, as described above.

In embodiments, global assessment tests may include assessments such as, but not limited to Clinician’s Interview-Based Impression of Change plus caregiver assessment (the CIBIC-plus), and Clinical Dementia Rating-sum of boxes (CDR-SB).

In embodiments, cognitive tests may include assessments such as, but not limited to, cognitive subscale of the Alzheimer’s disease Assessment Scale (ADAS-cog), and Mini Mental State Examination (MMSE).

In embodiments, functional tests may include assessments such as, but not limited to, severe impairment battery (SIB), modified Alzheimer’s disease cooperative study -activities of daily living inventory (ADCS-ADL) and modified Alzheimer’s disease cooperative study activities of daily living inventory for severe Alzheimer’s disease (ADCS-ADL-severe), Progressive Deterioration Scale (PDS), Instrumental Activities of Daily Living (IADL), and Katz activities of daily living (ADL) index.

In embodiments, behavioral and mood tests may include assessments such as, but not limited to, Neuropsychiatric Inventory (NPI).

At step 820, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 805.

At step 825, the patient is infused with pre-beta HDL particles in accordance with a therapy protocol. In an optional embodiment, a blood fraction is withdrawn from the patient presenting with AD. In an alternative embodiment, a blood fraction is obtained withdrawing blood from a person other than the patient. Therefore, the plasma obtained as a result of the blood fractionation process may be either autologous or non-autologous.

The blood fraction is subsequently treated, using the delipidation process described above to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

At optional step 830, the therapy protocol may be titrated or modulated up or down based on a therapeutic endpoint. In embodiments, one or more intra-treatment severity level assessments are made using diagnostic and/or cognitive procedures/tests. The one or more intra-treatment severity level assessments are made at predetermined points in time during the course of therapy. If the intra-treatment severity level assessments show a delay in the onset of additional symptoms, a halting in the worsening of symptoms, or an improvement in the patient’s condition, it is considered to be of therapeutic benefit. In embodiments, when therapeutic benefit is shown, the therapeutic amount may be titrated down wherein parameters such as, but not limited to, the dose range, frequency or cycle of treatment and/or course of therapy may be reduced. Alternately, the therapy protocol may be titrated up depending on various factors. Still alternately, if the intra-treatment severity level assessments show or do not show improvement in the patient’s condition, the therapy protocol is not modulated.

By way of example, for an early onset AD patient weighing 100 kg, where a dosage is determined to be 15 mg/kg, that patient will receive a dose of 1.5 g. It should be noted that if the patient presents with mild, moderate or severe AD, that dosage may be increased. The overall volume delivered to the patient via infusion therapy depends on the therapeutic product that is solubilized in a buffer or saline. For example, if the therapeutic product is autologous treated plasma, then the patient will receive a volume of therapeutic product equivalent to the volume that was extracted from the patient. If the therapeutic product is non-autologous treated plasma, the patient may receive a volume of 1L as one example. If the therapeutic product is non-autologous isolated, concentrated pre-beta particles, the volume may be much lower.

In an optional embodiment, which may be implemented in at least one treatment protocol as described in the present specification, a CETP inhibitor is used to increase HDL levels in a patient.

More specifically, a CETP inhibitor is in a class of compounds that inhibit cholesteryl ester transfer protein (CETP), which normally transfers cholesterol from HDL cholesterol to very low density or low density lipoproteins (VLDL or LDL). Inhibition of this process results in higher HDL levels and reduces LDL levels. Thus, in embodiments, the CETP inhibitor is used to increase the plasma level of HDL. The HDL (wherein the increased levels are created by the delipidation process which generates pre-beta HDL and/or by the use of a CETP inhibitor) will bind to the amyloid that may be present in plasma and/or the perivascular space/IPAD System/Perivascular Pathway. When bound to one or both of pre-beta HDL generated by the delipidation process or CETP-inhibited HDL, the amyloid may be transported (now in increased levels) to its side of degradation, thereby decreasing levels of amyloid. In embodiments, the degradation site is the liver. In embodiments, the CETP inhibitor binds to amyloid beta peptides in the peripheral vasculature of the patient, before the amyloid beta peptides have crossed the blood brain barrier and into the brain. In some embodiments, a CETP inhibitor is administered to the patient without administering pre-beta HDL particles, resulting in a decrease in flux of amyloid beta peptides into the brain. In other embodiments, a CETP inhibitor in administered to the patient in conjunction with the administration of pre-beta HDL particles, resulting in a decrease in flux of amyloid beta peptides into the brain (as peripheral amyloid beta peptides are bound by the CETP inhibitor and transported to a degradation site) and an increase in the removal of amyloid beta peptides from the brain (as amyloid beta peptide in the brain is bound by the pre-beta HDL particles, removed from the brain, and transported to a degradation site).

FIG. 8B is a flowchart describing a plurality of exemplary steps of a therapy protocol for treating an AD patient, in accordance with an embodiment of the present specification. At step 850, a patient first presents with a pathophysiological change that is consistent with early onset AD. Any of the aforementioned diagnostic techniques may be used in this step. In embodiments various biomarkers may be used to determine the pathophysiological change. For example, measuring the level of amyloid peptide (including 40 and 42) may be used to assess the extent of a pathophysiological change characteristic of AD. In embodiments, the ratio of beta amyloid peptide 42 to beta amyloid peptide 40 is measured to determine and assess the extent of a pathophysiological change characteristic of AD and to assess the efficacy of treatment. Particularly, the ratio of beta amyloid peptide 42 to beta amyloid peptide 40 in plasma and in cerebrospinal fluid is reduced in patients with AD. An AD patient may present with a ratio of beta amyloid peptide 42 to beta amyloid peptide 40 of approximately 0.057, while an individual without AD may have a ratio of 0.073. In an embodiment, the patient may present with cerebral amyloid angiopathy (CAA) as detected using imaging and/or biomarker techniques. For example, in embodiments, CAA patients could present with microbleeds and/or dilated perivascular space/IPAD System/Perivascular Pathways upon imaging and/or biomarker studies relative to individuals without CAA. A change in the incidence in microbleeds and/or size of the perivascular space/IPAD System/Perivascular Pathway may be used to assess efficacy of treatment.

At step 855, a patient who is diagnosed with AD is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

Spinal Fluid Test (detection of Beta Amyloid Fragments), is a diagnostic test that requires drawing fluid from the spinal region. Researchers have identified a protein “signature” in the spinal fluid of patients with CAA, which could represent an important advance in its diagnosis. The signature was found in the cerebrospinal fluid (CSF) of 90% of people with a diagnosis of Alzheimer’s disease and 72% of people with mild cognitive impairment (MCI) - a disorder that often progresses to Alzheimer’s. Researchers measured concentrations of three proteins previously identified as potential biological indicators, or biomarkers, for Alzheimer’s and MCI: amyloid-beta, tau, and phospho-tau. Alzheimer’s disease was identified in three independent study groups wherein the participants exhibited low levels of the amyloid protein amyloid-beta 1-42, along with high levels of total tau and elevated phospho-tau 181 (P-tau 181).

In embodiments, at optional step 860, the diagnosis and severity level of AD in the patient are additionally assessed based on one or more global, cognitive, functional, and behavioral measurements or tests, as described above.

In embodiments, global assessment tests may include assessments such as, but not limited to Clinician’s Interview-Based Impression of Change plus caregiver assessment (the CIBIC-plus), and Clinical Dementia Rating-sum of boxes (CDR-SB).

In embodiments, cognitive tests may include assessments such as, but not limited to, cognitive subscale of the Alzheimer’s disease Assessment Scale (ADAS-cog), and Mini Mental State Examination (MMSE).

In embodiments, functional tests may include assessments such as, but not limited to, severe impairment battery (SIB), modified Alzheimer’s disease cooperative study -activities of daily living inventory (ADCS-ADL) and modified Alzheimer’s disease cooperative study activities of daily living inventory for severe Alzheimer’s disease (ADCS-ADL-severe), Progressive Deterioration Scale (PDS), Instrumental Activities of Daily Living (IADL), and Katz activities of daily living (ADL) index.

In embodiments, behavioral and mood tests may include assessments such as, but not limited to, Neuropsychiatric Inventory (NPI).

At step 865 one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 850.

At step 870, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

At optional step 875, the therapy protocol may be titrated or modulated up or down based on a therapeutic endpoint.

In an optional embodiment, which may be implemented in at least one treatment protocol as described in the present specification, a CETP inhibitor is used in conjunction with the delipidation process described throughout the specification. In embodiments, the delipidation process of the present specification may be used in a short-term therapeutic approach (boosts), or intermittently, while the use of a CETP inhibitor may be used as a chronic, regular therapeutic approach. It should be noted that the steps shown FIG. 8A and FIG. 8B may be combined for an embodiment in which the CETP inhibitor is used in conjunction with the delipidation process. In embodiments, a combination therapy comprises the use of a CETP inhibitor as a chronic, regular therapeutic application with an intermittent application of pre-beta HDL particles.

FIG. 8C is a flowchart describing a plurality of exemplary steps of a therapeutic protocol for treating an AD patient using a CETP inhibitor and pre-beta HDL, in accordance with an embodiment of the present specification. At step 832, a patient first presents with a pathophysiological change that is consistent with early onset AD. Any of the aforementioned diagnostic techniques may be used in this step. In embodiments various biomarkers may be used to determine the pathophysiological change. For example, measuring the level of amyloid peptide (including 40 and 42) may be used to assess the extent of a pathophysiological change characteristic of AD. In embodiments, the ratio of beta amyloid peptide 42 to beta amyloid peptide 40 is measured to determine and assess the extent of a pathophysiological change characteristic of AD and to assess the efficacy of treatment. Particularly, the ratio of beta amyloid peptide 42 to beta amyloid peptide 40 in plasma and in cerebrospinal fluid is reduced in patients with AD. An AD patient may present with a ratio of beta amyloid peptide 42 to beta amyloid peptide 40 of approximately 0.057, while an individual without AD may have a ratio of 0.073. In an embodiment, the patient may present with cerebral amyloid angiopathy (CAA) as detected using imaging and/or biomarker techniques. For example, in embodiments, CAA patients could present with microbleeds and/or dilated perivascular space/IPAD System/Perivascular Pathways upon imaging and/or biomarker studies relative to individuals without CAA. A change in the incidence in microbleeds and/or size of the perivascular space/IPAD System/Perivascular Pathway may be used to assess efficacy of treatment.

At step 834, a patient who is diagnosed with AD is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

Spinal Fluid Test (detection of Beta Amyloid Fragments), is a diagnostic test that requires drawing fluid from the spinal region. Researchers have identified a protein “signature” in the spinal fluid of patients with CAA, which could represent an important advance in its diagnosis. The signature was found in the cerebrospinal fluid (CSF) of 90% of people with a diagnosis of Alzheimer’s disease and 72% of people with mild cognitive impairment (MCI) - a disorder that often progresses to Alzheimer’s. Researchers measured concentrations of three proteins previously identified as potential biological indicators, or biomarkers, for Alzheimer’s and MCI: amyloid-beta, tau, and phospho-tau. Alzheimer’s disease was identified in three independent study groups wherein the participants exhibited low levels of the amyloid protein amyloid-beta 1-42, along with high levels of total tau and elevated phospho-tau 181 (P-tau 181).

In embodiments, at optional step 836, the diagnosis and severity level of AD in the patient are additionally assessed based on one or more global, cognitive, functional, and behavioral measurements or tests, as described above.

In embodiments, global assessment tests may include assessments such as, but not limited to Clinician’s Interview-Based Impression of Change plus caregiver assessment (the CIBIC-plus), and Clinical Dementia Rating-sum of boxes (CDR-SB).

In embodiments, cognitive tests may include assessments such as, but not limited to, cognitive subscale of the Alzheimer’s disease Assessment Scale (ADAS-cog), and Mini Mental State Examination (MMSE).

In embodiments, functional tests may include assessments such as, but not limited to, severe impairment battery (SIB), modified Alzheimer’s disease cooperative study -activities of daily living inventory (ADCS-ADL) and modified Alzheimer’s disease cooperative study activities of daily living inventory for severe Alzheimer’s disease (ADCS-ADL-severe), Progressive Deterioration Scale (PDS), Instrumental Activities of Daily Living (IADL), and Katz activities of daily living (ADL) index.

In embodiments, behavioral and mood tests may include assessments such as, but not limited to, Neuropsychiatric Inventory (NPI).

At step 838 one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 832.

At step 840, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

It should be noted that in various embodiments of the present specification, administration of a CETP inhibitor is provided on a chronic basis as described above.

At step 842, the patient is infused with pre-beta HDL particles in accordance with a therapy protocol. In an optional embodiment, a blood fraction is withdrawn from the patient presenting with AD. In an alternative embodiment, a blood fraction is obtained withdrawing blood from a person other than the patient. Therefore, the plasma obtained as a result of the blood fractionation process may be either autologous or non-autologous.

The blood fraction is subsequently treated, using the delipidation process described above to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

It should be noted that in various embodiments of the present specification, administration of pre-beta HDL particles, as described in step 842, is performed on an intermittent basis during chronic administration of a CETP inhibitor, as described in step 840.

At optional step 844, the therapy protocol may be titrated or modulated up or down based on a therapeutic endpoint.

It is known that two key functions of the brain include a) supplying blood via the vascular lumen and b) elimination of interstitial fluids (ISF) and soluble metabolites, such as amyloid beta (Aβ) from the brain and retina via arterial walls. The Intramural Peri-Arterial Drainage (IPAD) system is comprised of basement membranes in the walls of capillaries and arteries and is used to enable fluid drainage toward peripheral lymph nodes. In embodiments, infusion of preβ-HDL provides a bi-modal neurovascular approach to treating AD. Pre-β HDL restores vascular wall biology resulting in improvements of tissue perfusion, facilitates the drainage of soluble oligomers, which include, but are not limited to Aβ and tau proteins present in the Intramural Peri-Arterial Drainage (IPAD) pathway, improves homeostasis, reduces inflammation, and thereby improves overall neuronal function. Embodiments of the present specification provide the ability to turbocharge metabolism and clearance into the circulation of harmful metabolites such as, but not limited to, soluble Aβ oligomers, Tau oligomers, and other soluble oligomers from the IPAD pathway and is therefore distinct from current approaches that target parenchymal Aβ plaques such as Aβ Immunotherapies. Removal of deposits containing soluble Aβ oligomers, Tau oligomers, and other soluble oligomers in accordance with the present specification, rather than targeting removal of parenchymal Aβ plaques, neutralizes neurotoxic effects. The metabolism of cerebral lipoproteins is largely separate from the periphery, although it appears that ApoA-I may gain access into the brain or signals across the endothelium to reduce neuro-inflammation and CAA, most likely by facilitating the drainage of lipoproteins into the blood or along the walls of arteries as IPAD. The expression of amyloid-precursor protein remains unaltered, with no effect on the synthesis of Aβ. The bi-modal neurovascular approach to AD treatment, in accordance with the embodiments, has the potential to slow/halt and even reverse disease progression and improve cognition in patients with mild to moderate AD. Serial treatment with preβ-HDL therapy in combination with Aβ immunotherapy are likely to succeed where Aβ immunotherapies alone have failed, because preβ-HDL therapy facilitates clearance of Aβ along the IPAD and into general circulation.

### Therapeutic Endpoints or Objectives for Alzheimer’s Disease and Cerebral Amyloid Angiopathy

In various embodiments, an AD patient’s baseline, starting or initial severity level is diagnosed/assessed and categorized as, one of early onset, mild, moderate or severe as described above. The baseline, starting or initial severity level refers to the severity of AD before the patient is treated with the pre-beta HDL and/or isolated pre-β HDL therapy of the present specification.

In various embodiments, a CAA patient’s baseline, starting or initial severity level is diagnosed/assessed and categorized as, one of early onset, mild, moderate or severe as described above. The baseline, starting or initial severity level refers to the severity of CAA before the patient is treated with the pre-beta HDL and/or isolated pre-β HDL therapy of the present specification.

In embodiments, the baseline, starting or initial severity level is diagnosed/assessed using at least one physiological diagnostic or advanced medical imaging technique. In some embodiments, the baseline, starting or initial severity level is additionally assessed by at least one global, cognitive, functional, behavioral measurement or test.

Magnetic Resonance Imaging (MRI) may be considered a neuroimaging examination for diagnosis and assessment of Alzheimer’s disease because it allows for accurate measurement of the 3-dimensional (3D) volume of brain structures, and in particular, the size of the hippocampus and related regions. HDL levels are inversely correlated with the development of AD in epidemiology studies. Elderly patients with higher levels of HDL tend to have a higher hippocampal volume. Lower hippocampal volume has been used as an index of AD disease progression. In addition, people with Down syndrome, and more specifically as exhibited in children, exhibit a lower hippocampal volume which is associated with cognitive impairment and a higher rate of developing AD than those that do not have Down syndrome. In an embodiments, a therapeutic benefit is recognized when the patient’s hippocampal volume is increased.

In an embodiment, a therapeutic benefit is recognized when a patient is able to maintain or be stabilized in their current state when treated with a therapy protocol of the present specification.

In an embodiment, a therapeutic benefit is recognized when a patient maintains/stabilizes symptoms when treated with a therapy protocol of the present specification when compared to a placebo.

In an embodiment, a therapeutic benefit is recognized when a patient shows a delay or halting of worsening of symptoms when treated with a therapy protocol of the present specification when compared to a placebo.

In an embodiment, a therapeutic benefit is recognized when a patient shows a delay in the rate of progression of symptoms when treated with a therapy protocol of the present specification when compared to a placebo.

In an embodiment, a therapeutic benefit is recognized when a patient shows an improvement in symptoms when treated with a therapy protocol of the present specification when compared to a placebo.

In some embodiments, after at least one treatment session or determinable time period at the end of which an effect of said at least one treatment session is amenable to measurement, the patient experiences a decrease in the accumulation of amyloid plaque in the perivascular space/IPAD System/Perivascular Pathway.

In some embodiments, after at least one treatment session or determinable time period at the end of which an effect of said at least one treatment session is amenable to measurement, the patient experiences an increase in the ratio of beta amyloid peptide 42 to beta amyloid peptide 40 in plasma relative to the ratio of beta amyloid peptide 42 to beta amyloid peptide 40 in plasma before treatment.

In some embodiments, after at least one treatment session or determinable time period at the end of which an effect of said at least one treatment session is amenable to measurement, the patient experiences an increase in the ratio of beta amyloid peptide 42 to beta amyloid peptide 40 in plasma in a range of 1% to 30%, and preferably by at least 15%, relative to the ratio of beta amyloid peptide 42 to beta amyloid peptide 40 in plasma before treatment.

In some embodiments, after at least one treatment session or determinable time period at the end of which an effect of said at least one treatment session is amenable to measurement, the patient experiences an increase in the ratio of beta amyloid peptide 42 to beta amyloid peptide 40 in cerebrospinal fluid relative to the ratio of beta amyloid peptide 42 to beta amyloid peptide 40 in cerebrospinal fluid before treatment.

In some embodiments, after at least one treatment session or determinable time period at the end of which an effect of said at least one treatment session is amenable to measurement, the patient experiences an increase in the ratio of beta amyloid peptide 42 to beta amyloid peptide 40 in cerebrospinal fluid in a range of 1% to 30%, and preferably by at least 15%, relative to the ratio of beta amyloid peptide 42 to beta amyloid peptide 40 in cerebrospinal fluid before treatment.

In some embodiments, after at least one treatment session or determinable time period at the end of which an effect of said at least one treatment session is amenable to measurement, the patient experiences a decrease in the incidence of microbleeds visualized on imaging and/or biomarker studies relative to the incidence of microbleeds visualized on imaging and/or biomarker studies before treatment, indicative of an improvement in, or stabilization of, symptoms.

In some embodiments, after at least one treatment session or determinable time period at the end of which an effect of said at least one treatment session is amenable to measurement, the patient experiences a decrease in the incidence of microbleeds visualized on imaging and/or biomarker studies by at least 1% relative to the incidence of microbleeds visualized on imaging and/or biomarker studies before treatment, indicative of an improvement in, or stabilization of, symptoms.

In some embodiments, after at least one treatment session or determinable time period at the end of which an effect of said at least one treatment session is amenable to measurement, the patient experiences a decrease in a size of a perivascular space/IPAD System/Perivascular Pathway visualized on imaging and/or biomarker studies relative to a size of a perivascular space/IPAD System/Perivascular Pathway visualized on imaging and/or biomarker studies before treatment, indicative of an improvement in, or stabilization of, symptoms.

In some embodiments, after at least one treatment session or determinable time period at the end of which an effect of said at least one treatment session is amenable to measurement, the patient experiences a decrease in a size of a perivascular space/IPAD System/Perivascular Pathway visualized on imaging and/or biomarker studies by at least 1% relative to a size of a perivascular space/IPAD System/Perivascular Pathway visualized on imaging and/or biomarker studies before treatment, indicative of an improvement in, or stabilization of, symptoms.

Following are a plurality of non-limiting, exemplary therapeutic endpoints with reference to the baseline, starting or initial severity level:

In embodiments, after at least one treatment session or determinable time period at the end of which an effect of said at least one therapy treatment is amenable to measurement, the rate of progression, level or amount of a patient’s physiological and/or cognitive parameter, is unchanged relative to the rate, level or amount of that patient’s physiological and/or cognitive parameter before therapy treatment.

In embodiments, after at least one treatment session or determinable time period at the end of which an effect of said at least one therapy treatment is amenable to measurement, the rate of progression, level or amount of a patient’s physiological and/or cognitive parameter, is delayed relative to the rate, level or amount of that patient’s physiological and/or cognitive parameter before therapy treatment.

In embodiments, after at least one treatment session or determinable time period at the end of which an effect of said at least one therapy treatment is amenable to measurement, the rate of progression, level or amount of a patient’s physiological and/or cognitive parameter, is modified relative to the rate of progression, level or amount of that patient’s physiological and/or cognitive parameter before therapy treatment.

In embodiments, after at least one treatment session or determinable time period at the end of which an effect of said at least one therapy treatment is amenable to measurement, the rate of progression, level or amount of that patient’s physiological and/or cognitive parameter is improved relative to the rate of progression, level or amount of that patient’s physiological and/or cognitive parameter before therapy treatment.

In some embodiments, after at least one treatment session or determinable time period at the end of which an effect of said at least one treatment session is amenable to measurement, the patient experiences an improvement of the Mini-Mental Score Examination (MMSE) score relative to the MMSE score before treatment, indicative of an improvement in, or stabilization of, AD-related symptoms.

In some embodiments, after at least one treatment session or determinable time period at the end of which an effect of said at least one treatment session is amenable to measurement, the patient experiences an increase of the Mini-Mental Score Examination (MMSE) score in a range of 1-10 points, and preferably by at least 3 points, relative to the MMSE score before treatment, indicative of an improvement in, or stabilization of, AD-related symptoms.

In some embodiments, after at least one treatment session or determinable time period at the end of which an effect of said at least one treatment session is amenable to measurement, the patient experiences an improvement of the ADAS-cog score indicative of an improvement in, or stabilization of, AD-related symptoms.

In some embodiments, after at least one treatment session or determinable time period at the end of which an effect of said at least one treatment session is amenable to measurement, the patient experiences an improvement of the CIBIC-plus score indicative of an improvement in, or stabilization of, AD-related symptoms.

In some embodiments, after at least one treatment session or determinable time period at the end of which an effect of said at least one treatment session is amenable to measurement, the patient experiences an improvement of the SIB score indicative of an improvement in, or stabilization of, AD-related symptoms.

In some embodiments, after at least one treatment session or determinable time period at the end of which an effect of said at least one treatment session is amenable to measurement, the patient experiences an improvement of the ADCS-ADL score indicative of an improvement in, or stabilization of, AD-related symptoms.

In some embodiments, after at least one treatment session or determinable time period at the end of which an effect of said at least one treatment session is amenable to measurement, the patient experiences an improvement of the ADCS-ADL-severe score indicative of an improvement in, or stabilization of, AD-related symptoms.

In some embodiments, after at least one treatment session or determinable time period at the end of which an effect of said at least one treatment session is amenable to measurement, the patient experiences an improvement of any one of the global, cognitive, functional, or behavioral test scores indicative of an improvement in, or stabilization of, AD-related symptoms.

In some embodiments, after at least one treatment session or determinable time period at the end of which an effect of said at least one treatment session is amenable to measurement, the patient experiences a decrease in the accumulation of amyloid plaque in the perivascular space/IPAD System/Perivascular Pathway indicative of an improvement or stabilization of AD-related symptoms.

### Acute Stroke

An acute stroke is the sudden interruption of blood supply to the brain. It may be caused either by an abrupt blockage of arteries leading to the brain, in which case it is called Ischemic Stroke (IS); or by bleeding into brain tissue when a blood vessel bursts, when it is known as a Hemorrhagic Stroke (HS).

### Ischemic Stroke (IS)

IS is most commonly caused by narrowing of the arteries in the neck or head. The narrowing of the arteries is often caused by atherosclerosis or gradual cholesterol deposition. If the arteries become too narrow, blood cells may collect and form blood clots.

There are two main types of IS: Thrombotic Stroke (TS) and Embolic Stroke (ES).

### Thrombotic Stroke (TS)

A TS, also referred to as cerebral thrombosis or cerebral infarction, occurs when diseased or damaged cerebral arteries become blocked by the formation of a blood clot within the brain. TS may further be divided into additional categories that correlate to the location of the blockage within the brain:

### Carotid Atherosclerosis (CA)

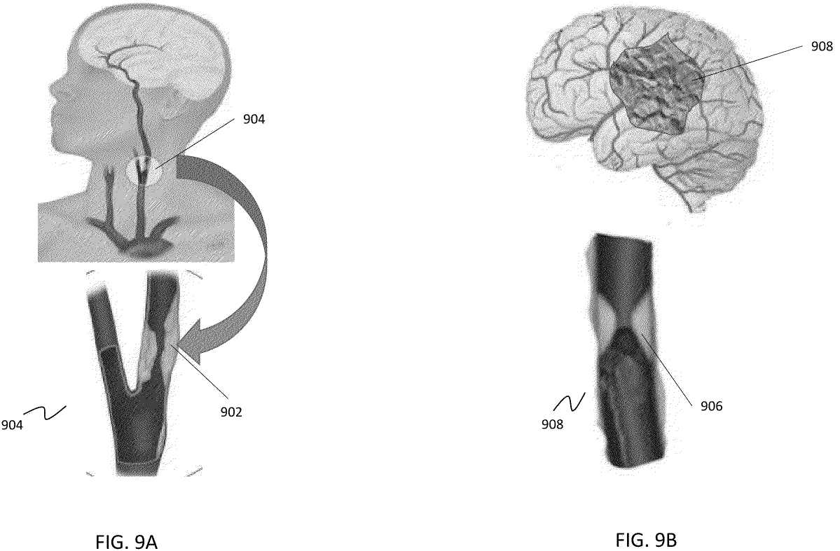

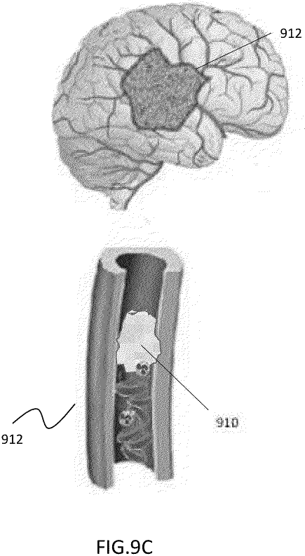

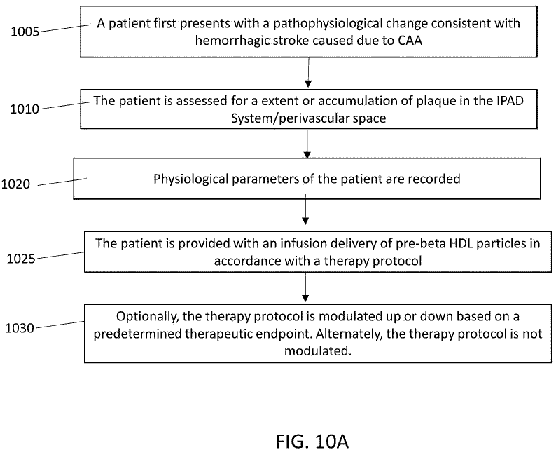

CA is said to occur when the blockage is in one of the brain’s larger blood-supplying arteries such as the carotid. Based on one or more tests, CA patients are identified as having plaque in the carotid artery. Sometimes the CA patients are asymptomatic. However, the affected artery may rupture in a manner similar to how plaque can rupture in a coronary artery. FIG. 9A illustrates plaque 902 in a carotid artery 904 of a patient. The illustrated blockage can potentially result in stroke, and is a candidate for therapy in accordance with the embodiments of the present specification.

### Cerebral Atherosclerosis

Cerebral atherosclerosis is said to occur when the blockage is in one of the brain’s larger blood-supplying arteries such as the middle cerebral. FIG. 9B illustrates plaque 906 in a middle cerebral artery 908 of a patient. The illustrated plaque deposit can potentially result in a stroke, and is a candidate for therapy in accordance with the embodiments of the present specification.

### Lacunar Stroke

Lacunar stroke is said to occur when one or more of the brain’s smaller but deeper penetrating arteries are blocked. This type of stroke is usually not recognized by patients as it destroys a very small part of the brain. This is sometimes also called arteriosclerosis. An MRI of patient’s brain which has suffered lacunar strokes may appear like Swiss cheese with little holes where these strokes have occurred. These strokes may result in losing function for a long period of time, known as a lacunar state or vascular dementia. Cholesterol plaques are considered to be a major risk factor for these strokes. Therefore patients with these strokes can be treated with the therapy in accordance with embodiments of the present specification.

### Embolic Stroke (ES)

An ES is also caused by a clot in an artery. However, in this case the clot (or emboli) forms somewhere other than in the brain itself. Often from the heart, these emboli will travel in the bloodstream until they become lodged and cannot travel any farther. This naturally restricts the flow of blood to the brain and results in near-immediate physical and neurological deficits. Sometimes the embolus formed by the breaking-off of plaque from carotid artery, and travels through the circulation to blood vessels in the brain. As the vessels become smaller, the emboli lodge themselves in the vessel wall and restrict blood flow in the brain. FIG. 9C illustrates an embolus 910 lodged within a central cerebral artery 912. The stroke caused by the emboli may result in temporary loss of function, or if the embolus is large, it could result in permanent loss of function. It can be treated by the therapy in accordance with embodiments of the present specification. Sometimes, an embolus formed as a result of a Hemorrhagic Stroke, resulting in ischemia as part of a section that is created as hemorrhage. The embolus can be treated as a symptom of Hemorrhagic Stroke (HS), discussed subsequently herein.

### Hemorrhagic Stroke (HS) and CAA

Protein deposits made from a material called amyloid, present in the perivascular space/IPAD System/Perivascular Pathway of blood vessels in the brain, may result in Cerebral Amyloid Angiopathy (CAA). In cases of severe CAA, the protein deposits cause blood vessels to crack, in which case blood leaks out and damages the brain, resulting in hemorrhagic stroke. These protein deposits are very similar to those found in the brain in Alzheimer’s disease (AD). While there may be other causes of hemorrhagic stroke, the presence of CAA is addressed by instances of the present specification, in a manner similar to therapy and treatment of AD. Sometimes, presence of CAA results in AD in association with hemorrhagic stroke, and may be addressed by embodiments of the present specification.

Hemorrhagic stroke is determined by physical examination, and may be confirmed by following diagnostic tests such as imaging. In hemorrhagic stroke, the blood that leaks out of a vessel damaged by CAA can cause the surrounding region of the brain to suddenly stop working properly, resulting in symptoms like weakness or paralysis of the limbs, difficulty speaking, loss of sensation or balance, or even coma. If blood leaks out to the sensitive tissue around the brain, it can cause a sudden and severe headache. Other symptoms sometimes caused by irritation of the surrounding brain are seizures (convulsions) or short spells of temporary neurologic symptoms such as tingling or weakness in the limbs or face.

In embodiments, treatments and protocols of the present specification are applicable to patients exhibiting hemorrhagic stroke in the presence of CAA. In embodiments, a baseline, starting or initial severity level of HS is diagnosed/assessed using at least one physiological diagnostic or advanced medical imaging technique. For example, measuring the level of amyloid peptide may be used to assess a possible treatment benefit.

In some cases, diagnostic imaging tests are used to determine the accumulation or regional lesions of plaque in the perivascular space/IPAD System/Perivascular Pathway. The advanced medical imaging techniques are used to both determine the extent of plaque in the perivascular space/IPAD System/Perivascular Pathway and to assess a severity level of Alzheimer’s disease. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

A specific Amyloid Positron Emission Tomography (PET) Scan, also referred to as Amyloid PET imaging, represents a potential major advance in diagnosis of HS and/or an assessment of the degree of impairment. The scan visualizes plaque regions or lesions present in the brain, which are prime suspects in damaging the vessels and causing leakage of blood into the brain. The scan technique employs radioactive tracers to highlight amyloid protein plaque regions or lesions within the brain, which are a hallmark of CAA. Amyloid PET scanning enables the “illumination” of amyloid plaques on a brain PET scan, enabling accurate detection of plaques in living people. The scan may allow for a diagnosis or assessment of HS, prior to the presentation of symptomatology.

It should be noted that persons with any combination of independent risk factors, such as but not limited to different levels of certain ApoE alleles as described above, high overall serum total cholesterol levels, and high blood pressure have an amplified risk of developing HS at some point in their lifetime. Accordingly, research has suggested that lowering serum cholesterol levels may reduce a person’s risk for HS.

In aspects of the present specification, a treated plasma that contains pre-beta HDL particles with reduced lipid content is delivered to the patient via infusion therapy. The process is designed such that HDL particles are treated to reduce their lipid levels and yield pre-beta HDL particles without destruction of plasma proteins or substantially affecting LDL particles.

The HDL lipoprotein particles are comprised of ApoA-I, phospholipids and cholesterol. Persons of ordinary skill in the art would appreciate that Apolipoprotein A-I (ApoA-I) particles comprise of two sub-fractions, pre-β HDL and α-HDL, which have pre-beta and alpha electrophoretic mobility, respectively. Thus, pre-β HDL represents ApoA-I molecules complexed with phospholipids.

In aspects of the present specification, a treated plasma that contains pre-beta HDL particles with reduced lipid content is delivered to the patient via infusion therapy. In an embodiment, the pre-beta high density lipoproteins have a concentration of alpha high density lipoproteins in addition to the pre-beta high density lipoproteins from the blood fraction prior to mixing.

In aspects of the present specification, isolated pre-β HDL particles are infused into the patient’s blood stream to bind to beta amyloid particles and clear the cerebral IPAD System/Perivascular Pathway. Referring back to FIGS. 5, 6, 7A, 7B, and 7C, the illustrations for removal of beta amyloid particles in accordance with embodiments of the present specification are explained, and are also applicable to removal of beta amyloid particles in cases of HS when CAA is present.

To generate and subsequently infuse the patient with treated plasma or with a solution containing an increased concentration of isolated pre-β HDL, a blood fraction is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

In some alternate embodiments, Low Density Lipoprotein (LDL) is also separated from the plasma. Separated LDL is usually discarded. In alternative embodiments, LDL is retained in the plasma. In accordance with embodiments of the present specification, the resultant blood fraction includes plasma with HDL, and may or may not include other protein particles.

In one embodiment, the process of blood fractionation is performed by withdrawing blood from the patient presenting with HS, and who is being treated by the physician. In an alternative embodiment, the process of blood fractionation is performed by withdrawing blood from a person other than the patient. Therefore, the plasma obtained as a result of the blood fractionation process may be either autologous or non-autologous.

In an optional embodiment, the autologous or non-autologous plasma obtained is subjected to a delipidation process as described in greater detail above with respect to FIG. 1 but repeated briefly herein. The resultant blood fraction is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. The plasma and solvent are introduced into at least one apparatus for mixing, agitating, or otherwise contacting the plasma with the solvent. The plasma may be transported using a continuous or batch process. The solvents dissolve lipids from the plasma. In embodiments of the present specification, the solvents dissolve lipids to yield treated plasma that contains pre-beta HDL particles with reduced lipid content. The process is designed such that HDL particles are treated to reduce their lipid levels and yield pre-beta HDL particles without destruction of plasma proteins or substantially affecting LDL particles. The resultant treated plasma containing pre-beta HDL particles with reduced lipid content, which was separated from the solvents, is treated appropriately and may subsequently be returned to the patient in an embodiment.

In an optional embodiment, the resultant fluid containing pre-beta HDL particles is further processed, in a second stage, to separate or to isolate pre-β HDL particles. In an embodiment, the second stage occurs in a separate and discrete area from the delipidation process. In an alternate embodiment, the second stage processing occurs in-line with the delipidation system, whereby the system may be connected to an affinity column sub-system or ultracentrifugation sub-system. The resultant separated pre-β HDL particles may then be introduced to the bloodstream of the patient as described below.

### Therapeutic Protocols for Administering Pre-Beta HDL Particles and CETP Inhibitors for HS

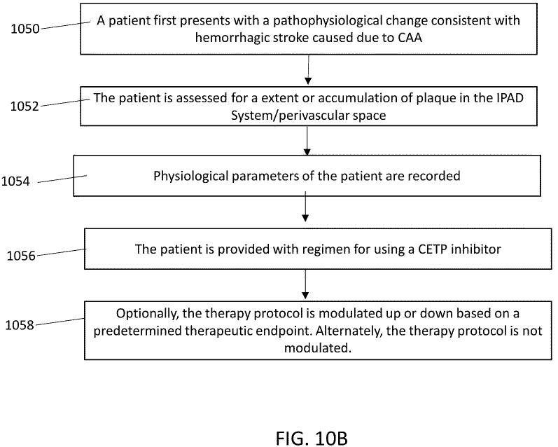

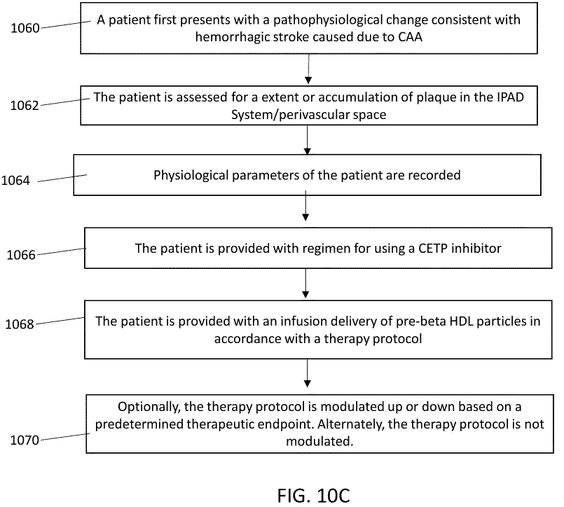

FIG. 10A is a flowchart describing a plurality of exemplary steps of a therapy protocol for treating an HS patient, in accordance with an embodiment of the present specification. At step 1005, a patient first presents with a pathophysiological change that is consistent with potential for HS. Any of the aforementioned diagnostic techniques may be used in this step. In embodiments various biomarkers may be used to determine the pathophysiological change. For example, measuring the level of amyloid peptide may be used to assess the extent of a pathophysiological change characteristic of HS. In an embodiment, the patient may present with cerebral amyloid angiopathy (CAA) as detected using a diagnostic imaging technique. At step 1010, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

At step 1020, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 1005.

At step 1025, the patient is infused with pre-beta HDL particles in accordance with a therapy protocol. In an optional embodiment, a blood fraction is withdrawn from the patient presenting with the CAA. In an alternative embodiment, a blood fraction is obtained withdrawing blood from a person other than the patient. Therefore, the plasma obtained as a result of the blood fractionation process may be either autologous or non-autologous. The blood fraction is subsequently treated, using the delipidation process described above to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

At optional step 1030, the therapy protocol may be titrated or modulated up or down based on a therapeutic endpoint. In embodiments, one or more intra-treatment severity level assessments are made using diagnostic and/or cognitive procedures/tests. The one or more intra-treatment severity level assessments are made at predetermined points in time during the course of therapy. If the intra-treatment severity level assessments show a delay in the onset of additional symptoms, a halting in the worsening of symptoms, or an improvement in the patient’s condition, it is considered to be of therapeutic benefit. In embodiments, when therapeutic benefit is shown, the therapeutic amount may be titrated down wherein parameters such as, but not limited to, the dose range, frequency or cycle of treatment and/or course of therapy may be reduced. Alternately, the therapy protocol may be titrated up depending on various factors. Still alternately, if the intra-treatment severity level assessments show or do not show improvement in the patient’s condition, the therapy protocol is not modulated.

FIG. 10B is a flowchart describing a plurality of exemplary steps of a therapy protocol for treating an HS patient, in accordance with an embodiment of the present specification. At step 1050, a patient first presents with a pathophysiological change that is consistent with potential for HS. Any of the aforementioned diagnostic techniques may be used in this step. In embodiments various biomarkers may be used to determine the pathophysiological change. For example, measuring the level of amyloid peptide may be used to assess the extent of a pathophysiological change characteristic of HS. In an embodiment, the patient may present with cerebral amyloid angiopathy (CAA) as detected using a diagnostic imaging technique. At step 1052, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

At step 1054, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 1050.

At step 1056, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

At optional step 1058, the therapy protocol may be titrated or modulated up or down based on a therapeutic endpoint.

In an optional embodiment, which may be implemented in at least one treatment protocol as described in the present specification, a CETP inhibitor is used in conjunction with the delipidation process described throughout the specification. In embodiments, the delipidation process of the present specification may be used in a short-term therapeutic approach (boosts), or intermittently, while the use of a CETP inhibitor may be used as a chronic, regular therapeutic approach. It should be noted that the steps shown FIG. 10A and FIG. 10B may be combined for an embodiment in which the CETP inhibitor is used in conjunction with the delipidation process. In embodiments, a combination therapy comprises the use of a CETP inhibitor as a chronic, regular therapeutic application with an intermittent application of pre-beta HDL particles.

FIG. 10C is a flowchart describing a plurality of exemplary steps of a therapeutic protocol for treating a patient of hemorrhagic stroke in the presence of CAA using a CETP inhibitor and pre-beta HDL particles, in accordance with an embodiment of the present specification. At step 1060, a patient first presents with a pathophysiological change that is consistent with potential for HS. Any of the aforementioned diagnostic techniques may be used in this step. In embodiments various biomarkers may be used to determine the pathophysiological change. For example, measuring the level of amyloid peptide may be used to assess the extent of a pathophysiological change characteristic of HS. In an embodiment, the patient may present with cerebral amyloid angiopathy (CAA) as detected using a diagnostic imaging technique. At step 1062, a patient who is diagnosed with CAA is monitored to determine an extent of accumulation of plaque in the perivascular space/IPAD System/Perivascular Pathway, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

At step 1064, one or more physiological parameters of the patient are recorded. In embodiments, the one or more physiological parameters are those that may be incidental to determining one or more therapy parameters. For example, the patient’s weight is recorded to determine a dosing range for the patient. It should be appreciated that the physiological parameters may be first recorded prior to step 1060.

At step 1066, the patient is provided with a regimen for using a CETP inhibitor. In an embodiment, the dose ranges from 1 mg to 1000 mg, and any increment therein. In embodiments, a patient may be given a CETP inhibitor at any interval that will achieve the desired therapeutic outcome, which includes, but is not limited to twice daily, weekly, biweekly, monthly, every two months, every six months, yearly or any increment therein. In embodiments, a patient is given a CETP inhibitor once daily. In an embodiment, a preferred dosage rate is once daily, given orally. In an embodiment, a preferred dosage amount of a CETP inhibitor, such as evacetrapib, is selected from one of: 30 mg/d, 100 mg/d, or 500 mg/d. In embodiments, the dosage amount may be modified depending upon desired therapeutic outcome. In embodiments, the desired therapeutic outcome may be measured by determining changes in a patient’s mean baseline lipoprotein levels (HDL-C, LDL-C, and triglycerides).

It should be noted that in various embodiments of the present specification, administration of a CETP inhibitor is provided on a chronic basis as described above.

At step 1068, the patient is infused with pre-beta HDL particles in accordance with a therapy protocol. In an optional embodiment, a blood fraction is withdrawn from the patient presenting with AD. In an alternative embodiment, a blood fraction is obtained withdrawing blood from a person other than the patient. Therefore, the plasma obtained as a result of the blood fractionation process may be either autologous or non-autologous.

The blood fraction is subsequently treated, using the delipidation process described above to obtain treated plasma containing pre-beta HDL particles. The treated plasma is optionally processed further to generate a product with an increased concentration of isolated pre-β HDL.

In an embodiment, the therapy protocol comprises an infusion delivery of pre-beta HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour +/- 100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of pre-beta HDL particles over a predetermined time period at a rate of 999 mL/hour.

It should be noted that in various embodiments of the present specification, administration of pre-beta HDL particles, as described in step 1068, is performed on an intermittent basis during chronic administration of a CETP inhibitor, as described in step 1066.

At optional step 1070, the therapy protocol may be titrated or modulated up or down based on a therapeutic endpoint.

### Therapeutic Endpoints or Objectives

In various embodiments, a CAA patient’s baseline, starting or initial severity level is diagnosed/assessed and categorized as, one of early onset, mild, moderate or severe as described above. The baseline, starting or initial severity level refers to the severity of CAA before the patient is treated with the pre-beta HDL and/or isolated pre-β HDL therapy of the present specification.

In embodiments, the baseline, starting or initial severity level is diagnosed/assessed using at least one physiological diagnostic or advanced medical imaging technique.

In various embodiments, the therapeutic endpoints that are realized with CAA as described above are the same therapeutic endpoints for HS.

### Hereditary Cerebral Amyloid Angiopathy (HCAA) and Hereditary Cerebral Hemorrhage with Amyloidosis (HCHWA)

HCHWA is a neurological condition in which, like CAA, an abnormal protein (amyloid) builds up in the walls of the arteries of the brain (and less frequently, veins). The amyloid deposits can lead to strokes, seizures, neurological deficits, cognitive decline, and dementia. This disease is also age-related, and may onset from middle-age. This condition can be fatal in one’s sixties with some variations, resulting from continuous neurological decline. Like CAA, HCAA is an aging-related condition caused by deposits of amyloid proteins in the wall or perivascular space/IPAD System/Perivascular Pathway of blood vessels in a brain. Low levels of CAA may usually be harmless, however, severe CAA may lead to the protein deposits causing the blood vessels to crack, in which case the blood can leak out and damage the brain. There are many different types of HCAA, mostly distinguishable by their genetic cause and signs and symptoms that occur. The various types of hereditary cerebral amyloid angiopathy are named after the regions where they were first diagnosed.

The Dutch type of HCAA is the most common form. Stroke is frequently the first sign of the Dutch type and is fatal in about one third of people who have this condition. Survivors often develop dementia and have recurrent strokes. About half of individuals with the Dutch type who have one or more strokes will have recurrent seizures (epilepsy). People with the Flemish and Italian types of HCAA are prone to recurrent strokes and dementia. Individuals with the Piedmont type may have one or more strokes and typically experience impaired movements, numbness or tingling (paresthesias), confusion, or dementia.

The first sign of the Icelandic type of HCAA is typically a stroke followed by dementia. Strokes associated with the Icelandic type usually occur earlier than the other types, with individuals typically experiencing their first stroke in their twenties or thirties.

Strokes are rare in people with the Arctic type of HCAA, in which the first sign is usually memory loss that then progresses to severe dementia. Strokes are also uncommon in individuals with the Iowa type. This type is characterized by memory loss, problems with vocabulary and the production of speech, personality changes, and involuntary muscle twitches (myoclonus).

Two types of HCAA, known as familial British dementia and familial Danish dementia, are characterized by dementia and movement problems. Strokes are uncommon in these types. People with the Danish type may also have clouding of the lens of the eyes (cataracts) or deafness.

Mutations in the Apolipoprotein (APP) gene are the most common cause of HCAA. APP gene mutations cause the Dutch, Italian, Arctic, Iowa, Flemish, and Piedmont types of this condition. Mutations in the CST3 gene cause the Icelandic type. Familial British and Danish dementia are caused by mutations in the ITM2B gene. The APP gene provides instructions for making a protein called amyloid precursor protein. This protein is found in many tissues and organs, including the brain and spinal cord (central nervous system). Mutations in the APP, CST3, or ITM2B gene lead to the production of proteins that are less stable than normal and that tend to cluster together (aggregate). These aggregated proteins form protein clumps called amyloid deposits that accumulate in certain areas of the brain and in its blood vessels. The amyloid deposits, or plaques, damage brain cells, eventually causing cell death and impairing various parts of the brain.

A Modified Boston Criteria incorporates cortical superficial siderosis into the radiological diagnosis to determine a probability of CAA, and may also be used for diagnosing HCAA and HCHWA. The criteria comprises of combined clinical, imaging and pathological parameters. The criteria has four tiers:
Tier 1 represents definite CAA, and determined during a full post-mortem examination. The examination reveals lobar, cortical, or cortical/subcortical hemorrhage and pathological evidence of severe CAA.
Tier 2 represents probable CAA with supporting pathological evidence. This examination may not be post-mortem. Clinical data and pathological tissue (evacuated hematoma or cortical biopsy specimen) demonstrate a hemorrhage as mentioned above, and some degree of vascular amyloid deposition, indicative of CAA.
Tier 3 represents probable CAA. In this case pathological confirmation is not required. Patients of 55 years or older with an appropriate clinical history are considered. Additionally, MRI findings demonstrate multiple hemorrhages restricted to lobar, cortical, or corticosubcortical regions (cerebellar hemorrhages allowed) of varying sizes/ages without another cause. Alternatively, a single lobar, cortical, or corticosubcortical hemorrhage and focal (three or less sulci) or disseminated (more than three sulci) cortical superficial siderosis without another cause.
Tier 4 represents possible CAA. This is also applicable to patients of 55 years or older age with an appropriate clinical history. Additionally, MRI findings demonstrate a single, (more than three sulci) cortical superficial siderosis without another; or focal or disseminated cortical superficial siderosis without another cause.

In aspects of the present specification, a treated plasma that contains pre-beta HDL particles with reduced lipid content is delivered to the patient via infusion therapy. The process is designed such that HDL particles are treated to reduce their lipid levels and yield pre-beta HDL particles without destruction of plasma proteins or substantially affecting LDL particles.

The HDL lipoprotein particles are comprised of ApoA-I, phospholipids and cholesterol. Persons of ordinary skill in the art would appreciate that Apolipoprotein A-I (ApoA-I) particles comprise of two sub-fractions, pre-β HDL and α-HDL, which have pre-beta and alpha electrophoretic mobility, respectively. Thus, pre-β HDL represents ApoA-I molecules complexed with phospholipids.

In aspects of the present specification, a treated plasma that contains pre-beta HDL particles with reduced lipid content is delivered to the patient via infusion therapy. In an embodiment, the pre-beta high density lipoproteins have a concentration of alpha high density lipoproteins in addition to the pre-beta high density lipoproteins from the blood fraction prior to mixing.

In aspects of the present specification, isolated pre-β HDL particles are infused into the patient’s blood stream to bind to beta amyloid particles and clear the cerebral IPAD System/Perivascular Pathway.

Referring back to FIG. 1, the process of removing beta amyloid particles and clearing the cerebral IPAD System/Perivascular Pathway is explained from the step of diagnosing a patient with a cerebral disease to the step of delivering pre-beta HDL to the patient.

In aspects of the present specification, isolated pre-β HDL particles are infused into the patient’s blood stream to bind to beta amyloid particles and clear the cerebral IPAD System/Perivascular Pathway. Referring back to FIGS. 5, 6, 7A, 7B, and 7C, the illustrations for removal of beta amyloid particles in accordance with embodiments of the present specification are explained, and are also applicable to removal of beta amyloid particles in cases of HCAA and HCHWA when CAA is present.

The process of a therapy protocol for treating HCAA and HCHWA may be identical to that for treating CAA, as described in FIGS. 4A-4C.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

1. A method for delaying a progression of, stabilizing, or improving symptoms related to cerebral amyloid angiopathy (CAA) in a patient, comprising:
monitoring a pathophysiological change indicative of CAA in a patient;
based on said monitoring, determining if at least one of amyloid plaque, Tau oligomers, or other oligomers is present in at least one of a perivascular space, an intramural peri-arterial drainage (IPAD) system, or a perivascular pathway of the patient in excess of a predetermined threshold; and,
based on a presence of the at least one of the amyloid plaque, the Tau oligomers, or the other oligomers in at least one of the perivascular space, the IPAD system, or the perivascular pathway of the patient, determining a treatment protocol for the patient, wherein the treatment protocol comprises, at least in part, administering to the patient a high density lipoprotein composition derived from mixing a blood fraction with a lipid removing agent.

2. The method of claim 1, wherein the high density lipoprotein composition is adapted to facilitate a drainage of at least one of soluble beta amyloids, soluble Tau oligomers, or other soluble oligomers.

3. The method of claim 1, further comprising determining an amount of at least one of the amyloid plaque, the Tau oligomers, or the other oligomers in at least one of the perivascular space, the IPAD system, or the perivascular pathway.

4. The method of claim 3, further comprising using diagnostic imaging to determine at least one of the presence or the extent of the at least one of the amyloid plaque, the Tau oligomers, or the other oligomers in at least one of the perivascular space, the IPAD system, or the perivascular pathway of the patient.

5. The method of claim 1, wherein the drainage is facilitated via the patient’s intramural peri-arterial drainage (IPAD) pathway.

6. The method of claim 1, wherein the high density lipoprotein composition is derived by:
obtaining the blood fraction from the patient, wherein the blood fraction has high-density lipoproteins;
mixing the blood fraction with the lipid removing agent to yield pre-beta high-density lipoproteins;
separating the pre-beta high-density lipoproteins; and
delivering the pre-beta high-density lipoproteins to the patient.

7. The method of claim 6, further comprising:
connecting the patient to a device for withdrawing blood;
withdrawing blood from the patient; and
separating blood cells from the blood to yield the blood fraction containing high density lipoproteins and low density lipoproteins.

8. The method of claim 6, wherein the pre-beta high density lipoproteins have an increased concentration of pre-beta high density lipoproteins relative to the high density lipoproteins from the blood fraction prior to mixing.

9. The method of claim 6, wherein the pre-beta high density lipoproteins have a concentration of alpha high density lipoproteins in addition to pre-beta high density lipoproteins from the blood fraction prior to mixing.

10. The method of claim 6, wherein the high density lipoprotein composition derived from mixing the blood fraction with the lipid removing agent is delivered to the patient via infusion therapy in a dosage ranging from 1 mg/kg to 250 mg/kg.

11. The method of claim 6, wherein the high density lipoprotein composition derived from mixing the blood fraction of the patient with the lipid removing agent is delivered to the patient via infusion therapy at a rate of 999 mL/hour +/- 100 mL/hr.

12. The method of claim 1, wherein the pathophysiological change is indicated by an accumulation of plaque in at least one of the perivascular space, the IPAD system, or the perivascular pathway of the patient resulting in cerebral amyloid angiopathy.

13. The method of claim 1, further comprising determining a severity of CAA in the patient using at least one of global functioning, cognitive functioning, activities of daily living, or behavioral assessments.

14. The method of claim 1, wherein after administering to the patient the high density lipoprotein composition, the patient experiences a decrease in an accumulation of the at least one of the amyloid plaque, the Tau oligomers, or other oligomers in at least one of the perivascular space, the IPAD system, or the perivascular pathway.

15. The method of claim 1, wherein after administering to the patient the high density lipoprotein composition, a rate of degeneration of the patient’s physiological and/or cognitive parameters indicative of CAA decreases.

16. The method of claim 1, wherein after administering to the patient the high density lipoprotein composition, a rate of degeneration of the patient’s physiological and/or cognitive parameters indicative of CAA, slows down relative to a rate of degeneration of the patient’s physiological and/or cognitive parameters indicative of CAA before administering to the patient the high density lipoprotein composition.

17. The method of claim 1, wherein after administering to the patient the high density lipoprotein composition, the patient’s physiological and/or cognitive symptoms indicative of CAA improve relative to the patient’s physiological and/or cognitive symptoms indicative of CAA before administering to the patient the high density lipoprotein composition.

18. The method of claim 1, wherein the high density lipoprotein composition is derived by:
obtaining a blood fraction from an individual other than the patient, wherein the blood fraction has high-density lipoproteins;
mixing the blood fraction with the lipid removing agent to yield pre-beta high-density lipoproteins;
separating the pre-beta high-density lipoproteins; and
delivering the pre-beta high-density lipoproteins to the patient.

19. The method of claim 1, wherein the lipid removing agent is at least one of phenols, hydrocarbons, amines, ethers, esters, alcohols, halohydrocarbons, halocarbons, diisopropyl ether (DIPE), diethyl ether (DEE), n-butanol, ethyl acetate, dichloromethane, chloroform, isoflurane, sevoflurane, perfluorocyclohexanes, trifluoroethane, cyclofluorohexanol, or combinations thereof.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)
